(19) United States
(12) Patent Application Publication
(10) Pub. No.: US 2011/0293643 A1
(43) Pub. Date: Dec. 1, 2011

(54) COMPOUNDS AND METHODS FOR INHIBITING MMP2 AND MMP9

(76) Inventor: David S. Wilkes, Indianapolis, IN (US)

(21) Appl. No.: 13/201,413

(22) PCT Filed: Feb. 9, 2010
(86) PCT No.: PCT/US10/23585

§ 371 Date: Aug. 12, 2011

Related U.S. Application Data
(60) Provisional application No. 61/152,512, filed on Feb. 13, 2009.

(51) Int. Cl. A61K 31/38; A61P 37/06; A61K 31/336

(52) U.S. Cl. 424/184.1

(57) ABSTRACT

The present invention relates to specific inhibitors of MMP2 and MMP9 and their use in immunosuppression.

### CROSS-REFERENCE TO RELATED APPLICATIONS

This application claims the benefit under 35 U.S.C. §119(e) of U.S. Provisional Patent Application No. 61/152,512 filed Feb. 13, 2009 which provisional application is incorporated herein by reference in its entirety.

### STATEMENT OF GOVERNMENT INTEREST

This invention was made with government support under Grant No. NHLBI RO1 HL081350-03 awarded by the National Institutes of Health. The government has certain rights in this invention.

### BACKGROUND OF THE INVENTION

1. Field of the Invention

The present invention relates generally to matrix metalloproteinase (MMP) inhibitors and methods of their use. In particular, the invention relates to inhibitors of MMP2 and MMP9 and their use in immunosuppression.

2. Description of the Related Art

Specific interactions of cells within the extracellular matrix are critical for the normal function of organisms. Alterations of the extracellular matrix are carried out by a family of zinc-dependent endopeptidases called matrix metalloproteinases (MMPs). The alterations are carried out in various cellular processes such as organ development, ovulation, fetus implantation in the uterus, embryogenesis, wound healing, and angiogenesis. Massova, L; Kotra, L. P.; Fridman, R.; Mobashery, S., FASEBJ. 1998, 12, 1075; Forget, M.-A.; Desrosier, R. R.; Beliveau, R. Can., J. Physiol. Pharmacol. 1999, 77, 465-480. MMPs consist of five major groups of enzymes: gelatinases, collagenases, stromelysins, membrane-type MMPs and matrilysins. The activities of MMPs in normal tissue functions is strictly regulated by a series of complicated zymogen activation processes and inhibition by protein tissue inhibitors for matrix metalloproteinases (“TIMPs”). Forget, M.-A.; Desrosier, R. R.; Beliveau, R. Can., J. Physiol. Pharmacol. 1999, 77, 465-480; Brew, K.; Dinakarpandian, D.; Nagase, H., Biochim. Biophys. Acta 2000, 1477, 267-283. Westermarck, J.; Kahari, V. M., FASEB J. 1999, 13, 781-792. Excessive MMP activity, when the regulation process fails, has been implicated in cancer growth, tumor metastasis, angiogenesis in tumors, arthritis and connective tissue diseases, cardiovascular disease, inflammation and autoimmune diseases. Massova, L; Kotra, L. P.; Fridman, R.; Mobashery, S., FASEB J. 1998, 12, 1075; Forget, M.-A.; Desrosier, R. R.; Beliveau, R. Can., J. Physiol. Pharmacol. 1999, 77, 465-480; Nelson, A. R.; Fingleton, B.; Rothenberg, M. L.; Matrisian, L. M., J. Clin. Oncol. 2000, 18, 1135. Increased levels of activity for the human gelatinases MMP2 and MMP9 have been implicated in the process of tumor metastasis. Dalberg, K.; Eriksson, E.; Enberg, U.; Kjellman, M.; Backdahl, M., World J. Surg. 2000, 24, 334-340. Salo, T.; Liotta, L. A.; Tryggvason, K. J., Biol. Chem. 1983, 258, 3058-3063. Pyke, C; Ralfkiaer, E.; Huhtala, P.; Hurskainen, T.; Dano, K.; Tryggvason, K., Cancer Res. 1992, 52, 1336-1341. Dumas, V.; Kanitakis, J.; Charvat, S.; Euvrard, S.; Faure, M.; Claudy, A., Anticancer Res. 1999, 19, 2929-2938. As a result, select inhibitors of MMPs (e.g., MMP2 and MMP9) are highly sought.

Additionally, anomalous MMP2 levels have been detected in lung cancer patients, where it was observed that serum MMP2 levels were significantly elevated in stage 1V disease and in those patients with distant metastases as compared to normal sera values (Garbisa et al., 1992, Cancer Res., 53: 4548, incorporated herein by reference.). Also, it was observed that plasma levels of MMP9 were elevated in patients with colon and breast cancer (Zucker et al., 1993, Cancer Res. 53: 140 incorporated herein by reference).

It has been shown that the gelatinase MMPs are most intimately involved with the growth and spread of tumors. It is known that the level of expression of gelatinase is elevated in malignancies, and that gelatinase can degrade the basement membrane which leads to tumor metastasis. Angiogenesis, required for the growth of solid tumors, has also recently been shown to have a gelatinase component to its pathology. Furthermore, there is evidence to suggest that gelatinase is involved in plaque rupture associated with atherosclerosis. Other conditions mediated by MMPs are restenosis, MMP-mediated osteopenias, inflammatory diseases of the central nervous system, skin aging, tumor growth, osteoarthritis, rheumatoid arthritis, septic arthritis, corneal ulceration, abnormal wound healing, bone disease, proteinuria, aneurysmal aortic disease, degenerative cartilage loss following traumatic joint injury, demyelinating diseases of the nervous system, cirrhosis of the liver, glomerular disease of the kidney, premature rupture of fetal membranes, inflammatory bowel disease, periodontal disease, age related macular degeneration, diabetic retinopathy, proliferative vitreoretinopathy, retinopathy of prematurity, ocular inflammation, keratoconus, Sjogren's syndrome, myopia, ocular tumors, ocular angiogenesis/neo-vascularization and corneal graft rejection. For recent reviews, see: (1) Recent Advances in Matrix Metalloproteinase Inhibitor Research, R. P. Beckett, A. H. Davidson, A. H. Drummond, P. Huxley and M. Whittaker, Research Focus, Vol. 1, 16-26, (1996), (2) Curr. Opin. Ther. Patents (1994) 4(1): 7-16, (3) Curr. Medicinal Chem. (1995) 2: 743-762, (4) Exp. Opin. Ther. Patents (1995) 5(2): 1087-110, (5) Exp. Opin. Ther. Patents (1995) 5(12): 1287-1196. MMPs involvement in inflammatory processes has been reviewed in W. Parks et al., Nature Reviews: Immunology, 2004, 4:617-629.

Several competitive inhibitors of MMPs are currently known. These inhibitors of MMPs take advantage of chelation of the active site zinc for inhibition of activity. Because of this general property, these competitive inhibitors for MMPs impact many biological pathways dependent on zinc and are often toxic to the host, which has been a major impediment in their clinical use. Greenwald, R. A. Ann. N.Y. Acad. ScL 1999, 575, 413-419; (a) Michaelides, M. R.; Curtin, M. L. Curr. Pharm. Des. 1999, 5, 787-819. (b) Beckett, R. P.; Davidson, A. H.; Drummond, A. H.; Huxley, P.; Whittaker, M. Drug Disc. Today 1996, 1, 16-26. Accordingly, the use of inhibitors of MMP with greater selectivity for one or more specific MMPs than known competitive inhibitors would be advantageous. Such methods will preferably not include negative long-term side-effects.

Immunomodulators have been found to be useful for treating systemic autoimmune diseases, such as lupus erythematosus and diabetes, as well as immunodeficiency diseases. Further, immunomodulators may be useful for immunotherapy of cancer or to prevent rejections of foreign organs or other tissues in transplants, e.g., kidney, heart, or bone marrow.

Various immunomodulator compounds have been discovered, including FK506, muramylic acid dipeptide derivatives, levamisole, niridazole, oxysuran, flagyl, and others from the groups of interferons, interleukins, leukotrienes, corticosteroids, and cyclosporins. Many of these compounds have been found, however, to have undesirable side effects and/or high toxicity. New immunomodulator compounds are therefore needed to provide a wider range of immunomodulator function for specific areas with a minimum of undesirable side effects.

Therefore, given the toxicity of immunosuppressant drugs and MMP inhibitors, there remains a need in the art for methods and compounds for effective treatment of immune-mediated disorders where dysregulation of MMPs may be involved. The present invention provides this and other advantages.

### BRIEF SUMMARY OF THE INVENTION

One aspect of the present invention provides a method for reducing alloantigen-induced proliferation of T cells comprising, administering to a transplant patient a therapeutically effective amount of a compound of Formula I:

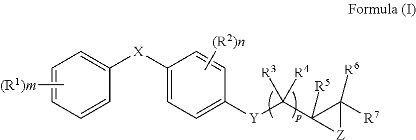

wherein:

m is 0, 1, 2, 3, 4 or 5;

n is 0, 1, 2, 3, 4 or 5;

p is 1, 2 or 3;

X is —O—, —S—, —CH₂— or a direct bond;

Y is —C(O)— or —S(O)₂—,

Z is —O— or —S—;

R¹ at each occurrence is the same or different and independently alkyl, alkenyl, aralkyl, haloalkyl, halogen, —OR⁸ or —NR⁹R¹⁰;

R² at each occurrence is the same or different and independently alkyl, alkenyl, aralkyl, haloalkyl, halogen, —OR⁸ or —NR⁹R¹⁰;

R³ and R⁴ are each the same or different and independently hydrogen or alkyl;

R⁵, R⁶ and R⁷ are each the same or different and independently hydrogen or alkyl;

R⁸ is hydrogen, alkyl, alkenyl, or aryl; and

R⁹ and R¹⁹ are each the same or different and independently hydrogen or alkyl;

or a pharmaceutically acceptable salt thereof.

In one embodiment of the methods of the invention, the compound of formula (I) is a compound of formula (Ia):

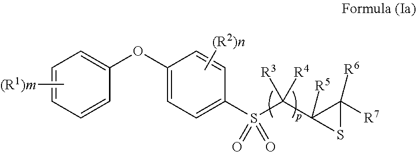

In a further embodiment of the methods of the present invention, the compound is SB-3CT

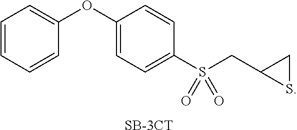

In yet further embodiments of the methods of the invention, the compound of formula (I) is a compound of formula (Ib) or (Ic):

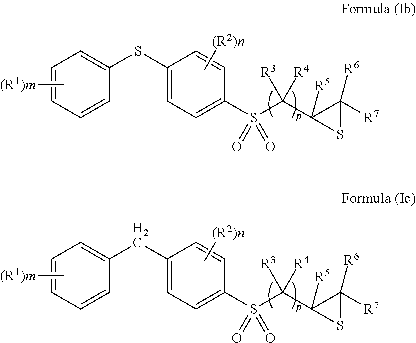

In certain embodiments of the methods of the invention, the transplant patient is a lung transplant patient. In another embodiment of the methods of the invention, the T cells are CD4+ T cells. In an additional embodiment, the methods further comprise administering prior to organ harvest, a therapeutically effective amount of a compound of Formula I to an organ donor donating an organ to the transplant patient.

Another aspect of the invention provides a method for inhibiting an immune response against a collagen in a transplant patient or a patient in need of a transplant comprising, administering to the patient a therapeutically effective amount of a compound of Formula I or a pharmaceutically acceptable salt thereof.

In certain embodiments, the compound of formula (I) is a compound of formula (Ia), (Ib) or (Ic) as described herein. In a further embodiment of the method, the compound is SB-3CT. In another embodiment, the transplant patient is a lung transplant patient.

Another aspect of the invention provides a method for improving the outcome of a transplant comprising, administering to a transplant patient a therapeutically effective amount of a compound of Formula I. In certain embodiments, the compound of formula (I) is a compound of formula (Ia), (Ib), (Ic) or SB-3CT. In one embodiment, the method further comprises administering prior to organ harvest, a therapeutically effective amount of a compound of Formula I to an organ donor donating an organ to the transplant patient. In certain embodiments of the method, the transplant patient is a lung transplant patient.

Yet another aspect of the invention provides a method for inhibiting an immune response in a patient in need thereof comprising, administering to the patient a therapeutically effective amount of a compound of Formula I or a pharmaceutically acceptable salt thereof. In one embodiment, the patient in need thereof has an autoimmune disease. In this regard, any autoimmune disease is contemplated herein, including but not limited to, alloimmune-induced autoimmunity post organ transplant (heart, lung, liver, kidney, pancreas, multi-visceral transplant, hematopoetic stem cell); collagen vascular diseases (systemic lupus erythematosus, rheumatoid arthritis, wegener's granulomatosis, scleroderma), multiple sclerosis, insulin dependent diabetes, celiac disease, inflammatory bowel disease, ulcerative colitis, Crohn's disease, systemic lupus erythematosus, psoriasis, and Insulin-dependent diabetes (type 1). In one particular embodiment, the patient in need thereof has asthma or a T cell mediated pulmonary disease. In certain embodiments, the immune response comprises a CD8+ T cell response or a CD4+ T cell response. In one embodiment, regulatory T cells are not inhibited by the compound of Formula I. In a further embodiment, the patient is a solid organ transplant patient.

Another aspect of the invention provides a method for reducing alloantigen-induced proliferation of T cells comprising, administering to a transplant patient a therapeutically effective amount of a compound that can selectively inhibit Matrix Metalloproteinase 2 and 9.

Yet a further aspect of the invention provides a method for inhibiting an immune response in a patient in need thereof comprising, administering to the patient a therapeutically effective amount of a compound that can selectively inhibit Matrix Metalloproteinase 2 and 9.

Another aspect of the invention provides a method for reducing the dosage of an immunosuppressant comprising administering to a patient in need thereof an effective amount of a compound of Formula I before or concurrent with administration of the immunosuppressant.

A further aspect of the invention provides a method for suppressing an immune response in a patient in need thereof comprising administering to the patient an effective amount of a compound of Formula I in combination with a known immunosuppressant (immunosuppressive drug). In this regard, any of a number of immunosuppressants may be used, such as, but not limited to, cyclosporin A, FK506, rapamycin, corticosteroids, purine antagonists (includes azathioprine and mycophenolate), campath, and anti-lymphocyte globulin.

Another aspect of the invention provides a method for reducing an immune response to Collagen V comprising administering to a patient in need thereof an effective amount of a compound of Formula I in combination with an effective amount of Collagen V, or a tolerizing fragment thereof. In one embodiment, the patient is a patient in need of a lung transplant or a lung transplant patient. In a further embodiment, the collagen V or tolerizing fragment thereof is administered orally or intravenously.

A further aspect of the present invention provides a composition for reducing alloantigen-induced proliferation of T cells in a transplant patient comprising, a therapeutically effective amount of a compound of Formula I where in certain embodiments, the compound of Formula (I) is (Ia), (Ib), (Ic), or SB-3CT as set forth herein. In certain embodiments, the composition is for reducing alloantigen-induced proliferation of T cells in a lung transplant patient. In a further embodiment, the T cells are CD4+ T cells. In certain embodiments, the composition is used prior to organ harvest in an organ donor donating an organ to the transplant patient.

Another aspect of the invention provides a composition for inhibiting an immune response against a collagen in a transplant patient or a patient in need of a transplant comprising, a therapeutically effective amount of a compound of Formula I where in certain embodiments, the compound of Formula (I) is (Ia), (Ib), (Ic), or SB-3CT as set forth herein. In one embodiment, the transplant patient is a lung transplant patient.

A further aspect of the invention provides a composition for improving the outcome of a transplant comprising, a therapeutically effective amount of a compound of Formula I where in certain embodiments, the compound of Formula (I) is (Ia), (Ib), (Ic), or SB-3CT as set forth herein. In this regard, in one embodiment, the composition is used in an organ donor prior to organ harvest. In certain embodiments, the transplant patient is a lung transplant patient.

Another aspect of the invention provides a composition for inhibiting an immune response in a patient in need thereof comprising, a therapeutically effective amount of a compound of Formula I where in certain embodiments, the compound of Formula (I) is (Ia), (Ib), (Ic), or SB-3CT as set forth herein. In certain embodiments, the patient in need thereof has an autoimmune disease selected from the group consisting of alloimmune-induced autoimmunity post organ transplant, collagen vascular diseases and multiple sclerosis. In one embodiment, the patient in need thereof has asthma or a T cell-mediated pulmonary disease. In certain embodiments, the T cell response is a CD8+ T cell response. In one particular embodiment, the CD8+ T cell response is an antigen-specific response. In a further embodiment, the immune response comprises a CD4+ T cell response, which may be a an antigen-specific response. In certain embodiments, the compositions do not inhibit regulatory T cells. In certain embodiments the composition is used in a solid organ transplant patient.

Yet a further aspect of the invention provides a composition for reducing alloantigen-induced proliferation of T cells comprising, a therapeutically effective amount of an agent that can selectively inhibit Matrix Metalloproteinase 2 and 9.

Another aspect of the invention provides a composition for inhibiting an immune response in a patient in need thereof comprising, a therapeutically effective amount of an agent that can selectively inhibit Matrix Metalloproteinase 2 and 9. In this regard the immune response may be an antigen-specific immune response.

Yet another aspect of the invention provides a composition comprising an effective amount of a compound of Formula I in combination with an immunosuppressant wherein the effective dosage of the immunosuppressant is reduced as compared to the effective dosage normally used in the absence of the compound of Formula I.

A further aspect of the invention is a composition for suppressing an immune response in a patient comprising an effective amount of a compound of Formula I in combination with a known immunosuppressant. In this regard, the immune response may be an antigen-specific immune response. In certain embodiments the known immunosuppressant may be, but is not limited to, one or more of cyclosporin A, FK506, rapamycin, corticosteroids, purine antagonists, campath and anti-lymphocyte globulin.

Another aspect of the invention is a composition for reducing an immune response to Collagen V comprising administering to a patient in need thereof an effective amount of a compound of Formula I in combination with an effective amount of Collagen V, or a tolerizing fragment thereof. In certain embodiments, the patient is a patient in need of a lung transplant or a lung transplant patient. In another embodiment, the collagen V or tolerizing fragment thereof is administered orally or intravenously.

Another aspect of the invention is a use of the compositions comprising the compound of Formula (I), where the compound may be that of Formula (Ia), (Ib), (Ic), or SB-3CT, in the manufacture of a medicament for reducing alloantigen-induced proliferation of T cells in a transplant patient, for inhibiting an immune response against a collagen in a transplant patient or a patient in need of a transplant, for improving the outcome of a transplant, for inhibiting an immune response in a patient in need thereof, or for reducing an immune response to collagen V in a patient in need thereof.

### BRIEF DESCRIPTION OF THE DRAWINGS

FIG. 1. Differential MMP9 mRNA and protein expression in CD4⁺ and CD8⁺ T cells. Pure splenic A) CD4⁺ and B) CD8⁺ T cells were cultured in the absence or presence of anti-CD3 antibody (1 μg/ml). RNA was isolated, cDNA synthesized and mRNA expression levels were measured by quantitative RT PCR. Data were normalized to β-actin. Data are representative of two separate experiments performed in triplicate. C) Gelatin zymogram analysis of CD4⁺ and CD8⁺ T cell lysates and supernatant. Data are representative of one of four separate experiments.

FIG. 2. Broad spectrum and specific MMP inhibition abrogated anti-CD3 induced T cell proliferation. Pure splenic CD4⁺ T cells were treated with A) 1,10 phenanthroline (0.001-0.1 μM) or B) COL-3 (1-100 μM). C) CD4⁺ and D) CD8⁺ T cells were treated with SB3CT (5-25 μM) or vehicle (DMSO⁺ PEG, diluted similarly in CRPMI) and cultured in the presence of anti-CD3 antibody (0.5 μg·ml) for 72 h. E) CD4⁺ and F) CD8⁺ SB3CT treated T cells cultured in the presence of anti-CD3 and exogenous murine IL-2 for 72 h. T cell proliferation was measured by 3H thymidine incorporation. Data are representative of the mean± of three experiments performed in triplicate. #p<0.05, *p<0.001.

FIG. 3. MMP2, MMP9 and MMP2/9 deficient CD4 T cells display altered proliferative ability. Wild-type and A) MMP2−/− CD4⁺, B) MMP9−/− CD4⁺, C) MMP2/9−/− CD4⁺, D) MMP9−/− CD8⁺ T cells were cultured in the presence of anti-CD3 antibody (0.5 μg/ml) for 72 h. T cell proliferation was measured by 3H thymidine incorporation. Data representative of the mean SD± of three separate experiments performed in triplicate. #p=0.02, *p=0.006, **p<0.001.

FIG. 4. MMP deficiency or inhibition decreases calcium flux. A) CD4⁺ or B) CD8⁺ T cells isolated from wild-type and MMP9−/− mice. C-D) CD8⁺ T cells were treated with SB3CT (10 μM) or vehicle (DMSO⁺ PEG, diluted similarly in CRPMI). A-C) Cells were cultured in calcium-free or D) calcium containing media and stimulated with anti-CD3 antibody (10 μg/ml). Calcium flux was measured for 100 seconds in real time. Data are representative of one of three separate experiments performed in triplicate.

FIG. 5. MMP deficiency or inhibition alters NFATc1 and CD25 expression. A-B) CD4⁺ T cells were isolated from wild-type, MMP2−/− and MMP9−/− mice. C-D) CD4⁺ T cells were treated with SB3CT (5-20 μM) or vehicle (DMSO⁺ PEG, diluted similarly in CRPMI). Cells were cultured in the presence or absence of anti-CD3 antibody (1 μg/ml). NFATc1 and CD25 expression levels were measured by quantitative RT PCR. Data are representative of three separate experiments performed in triplicate. #p<0.05, ##p<0.01, *p<0.001.

FIG. 6. MMP9 inhibition down-regulates IL-2 and IFN-γ expression in CD4⁺ and CD8⁺ T cells. A-B) CD4⁺ T cells were isolated from wild-type and MMP9−/− mice. C-D) Wild-type CD4⁺ T cells were treated with SB3CT (10 μM) or vehicle (DMSO⁺ PEG, diluted similarly in CRPMI) for various timepoints. E-F) CD8⁺ T cells were isolated from wild-type and MMP9−/− mice. G-H) CD8⁺ T cells were treated with SB3CT (10 μM) for various time-points. Cells were cultured in the absence or presence of anti-CD3 antibody (1 μg/ml). IL-2 and IFN-γ mRNA and protein expression was measured by quantitative RT PCR and cytometric bead assay, respectively. Data are representative of 3 separate experiments performed in triplicate. *p<0.001.

FIG. 7. MMP9 inhibition does not induce regulatory T cell function. Wild-type, MMP9−/− and SB3CT (10 μM) or vehicle (DMSO⁺ PEG, diluted similarly in CRPMI) treated CD4⁺ T cells were cultured in the absence or presence of anti-CD3 (0.5 μg/ml). A-B) Foxp3 expression was measured by quantitative RT PCR. C) Cell culture supernatants were collected and assayed for IL-10 protein expression by cytometric bead assay. D) CD4⁺25− or E) CD4⁺25⁺ T cells were treated with SB3CT (10 μM) and cultured at varying ratios with fresh CD4⁺25− T cells in the presence of anti-CD3 (0.5 μg/ml). Data from panels A and -C are representative of one experiment performed in triplicate. Data from panels D-E are representative of three separate experiments performed in triplicate. #p<0.01, *p<0.001.

FIG. 8. SB3CT treated antigen-specific T cells (OT-1) display impairment in proliferative ability. A) OTI Tg CD8⁺ T cells were treated with SB-3CT (5-20 μM) or vehicle (DMSO⁺ PEG, diluted similarly in CRPMI) and cultured in the presence of OVA-pulsed antigen presenting cells (APCs) for 72 hours. Data are representative of two separate experiments performed in triplicate. #p<0.05, *p<0.001 B) Seven days after adoptive transfer, BAL fluid from the CC10-OVA (CC10) or non-transgenic (B6) mice was analyzed and total cells present in the BAL were quantitated. C) neutrophils were stained with GR1 and analyzed by means of flow cytometry. **p<0.01 as compared to stimulated wild-type cells. n=10 mice (CC10) per treatment group and 5 control mice (B6) per treatment group.

FIG. 9. Murine model of antigen-specific CD8⁺ effector T cell mediated lung injury. A) CD8⁺ Thy1.1⁺ T cells were isolated from the lung mice following the adoptive transfer of SB3CT (10 μM) or vehicle (DMSO⁺ PEG, diluted similarly in CRPMI). B) CD25 expression in CD8⁺ Thy1.1⁺ T cells from the lungs of CC10-OVA mice. *p<0.01 n=9 mice (CC10) per treatment group and 5 control mice (B6) per treatment group.

FIG. 10. Schematic diagram of differences in T cell activation in response to MMP inhibition (SB3CT) or absence (MMP9 deficiency). Following TCR stimulation (1) under normal cell conditions, there is an up-regulation of many signaling events (2) including an increase in calcium flux (3, 4), which leads to the up-regulation of NFAT (5), CD25 (6) and IL-2 (8) expression thereby allowing for CD25 cell surface presentation and binding of IL-2, leading to cell activation. In the absence of MMP9, calcium influx is significantly elevated although NFAT expression in down-regulated. The decrease in NFAT expression in turn leads to a decrease in CD25 and IL2 expression, while the regulatory pathways, Foxp3 and IL-10, are up-regulated, thereby decreasing cell activation.

FIG. 11. Phenotypic analysis of CD4⁺ and CD8⁺ MMP9−/− T cells. Pure splenic A) CD4⁺ and B) CD8⁺ T cells were isolated from wild-type (solid line open histograms) and MMP9 deficient (shaded histograms) mice. Cells were cultured in the presence of anti-CD3 antibody (0.5 μg/ml) for 24 hours. Cells were collected and surface expression of CD45RO, CD69, CD25, CD44, CD40L, CD62L, CTLA-4 was analyzed by flow cytometry. Dashed line histograms represent isotype controls. Data are representative of one of two separate experiments.

### DETAILED DESCRIPTION OF THE INVENTION

The present invention centers on the unexpected discovery that MMP2 and MMP9 are present intracellularly in T cells and regulate T cell activation. Thus, the present invention provides methods for inhibiting immune responses by targeted inhibition of MMP2 and MMP9. The present invention relates generally to methods for inhibiting an immune response in a subject in need thereof by selectively inhibiting MMP2 and/or MMP9. In particular, the present invention relates to methods for inhibiting T cell responses by selectively inhibiting MMP2 and/or MMP9.

### Matrix Metalloproteinase 2 and 9

Elevated expression of MMP2 and MMP9 is often seen in invasive and tumorigenic cancers including colorectal tumors, gastric carcinoma, pancreatic carcinoma, breast cancer, oral cancer, melanoma, malignant gliomas, Chondrosarcoma, and gastrointestinal adenocarcinoma. Levels are also increased in malignant astrocytomas, carcinomatous meningitis, and brain metastases. MMPs promote tumor progression and metastasis in invasive cancers by degradation of basement membranes and interstitial connective tissues, both components of the ECM (ExtraCellular Matrix). Collagen IV is the major element of the ECM. Other elements of the ECM include laminin-5, proteoglycans, entactin, and osteonectin. MMP2 & MMP9 efficiently degrade collagen IV and laminin-5, thereby allowing metastatic cancerous cells to migrate through the basement membrane (see Kundu G C, Patil D P. MMP2 (matrix metallopeptidase 2 (gelatinase A, 72 kDa gelatinase, 72 kDa type IV collagenase) Atlas Genet Cytogenet Oncol Haematol. October 2005).

MMPs are also known to regulate matrix remodeling in many pulmonary diseases. Experiments in a Wistar-Kyoto rat model compared rats treated with the global MMP inhibitor COL-3 with induced ischemia reperfusion injury to rats treated with MMP inhibitors pre- and post-lung transplantation (Iwata, T. et al. 2008 Transplantation 85:417). The results showed the ischemia reperfusion injury induced growth-related oncogene/CINC-1-dependent neutrophil influx, and upregulated tumor necrosis factor-alpha. Induction of MMP2 and MMP9 (at 4 and 24 hours) was associated with antigenic collagen (V) detected in the bronchoalveolar lavage and lung interstitium. Treatment with COL-3 reduced inflammation factors and resulted in lower levels of antigenic collagen (V) in bronchoalveolar lavage. Inhibiting MMPs in the donor lung before lung harvest and in the recipient after transplantation improved oxygenation and diminished polymorphonuclear leukocyte influx into the isograft.

Evidence from the rat model of lung transplantation showed benefit of specific MMP inhibitors compared to a global inhibitor. In particular, experiments show tissue-inhibitors of metalloproteinases (TIMP-1 and TIMP-2) have differential effects on delayed hypersensitivity responses to donor antigens and type V collagen (an autoantigen involved in the rejection response) but neither affected the onset of rejection pathology. In contrast COL-3, a global MMP inhibitor suppressed delayed type hypersensitivity, but also local production of tumor necrosis factor-alpha and interleukin-1 beta. While COL-3 did not prevent rejection pathology, it did induce intragraft B cell hyperplasia that was suggestive of post-transplant proliferative disorder.

Prior to the present invention, the ability of MMPs to function intracellularly and regulate immune cell function were unknown. Nonspecifically blocking MMPs with a global MMP inhibitor in vivo down regulated alloantigen and autoantigen-induced T cell proliferation in a rat lung transplant model (Iwata, T., et al. 2008 Transplantation 85:417), suggesting MMP activity may be involved in the pathogenesis of the rejection response.

MMP2 and MMP9 amino acid and polynucleotide sequences are publically available in databases such as GENBANK or SWISSPROT. Representative sequences may be found in GENBANK accession numbers AK310314[gi:64692100], AK312711[gi:164690513], and SwissProt P08253 (01-FEB-1991, sequence version 2) (MMP2) and NM_{—}004994[gi:74272286], AAD37404[gi:5002294], and NP_{—}004985[gi:74272287] (MMP9). As would be recognized by the skilled artisan, these are representative sequences and other sequence variants of MMP2 and MMP9 may be found in any of a variety of public databases and are contemplated for targeted inhibition by the present invention.

Thus, the present invention provides methods and compounds for inhibiting MMP2 and MMP9. In particular, the present invention centers on the discovery that MMP2 and MMP9 are present intracellularly in T cells and regulate T cell activation. Further, the present invention provides MMP2- and MMP9-specific inhibitors that can be used as immunosuppressive drugs by their specific action of inhibiting alloantigen and autoantigen-induced T cell proliferation.

### General Description SB-3CT Derivatives

In general, MMP inhibitors suitable for the methods described herein typically have a structure comprising three segments: (1) a hydrophobic region (e.g., a biphenyl moiety) that interacts with the P1′ subsite, which is a large hydrophobic pocket; (2) a hydrogen bond donor region (e.g., a sulfone or carbonyl moiety) that binds amides on protein backbones via hydrogen bonds; and (3) an electrophilic region (e.g., a thiirane or epoxide ring) that is susceptible to nucleophilic addition and is capable of binding to Zn²⁺ at the active site via coordination bond.

Suitable MMP inhibitors include, for example, those described in WO06/036928, which reference is incorporated herein in its entirety.

### Formula (I)

In certain specific embodiments, compounds suitable for the methods described herein are represented by Formula (I).

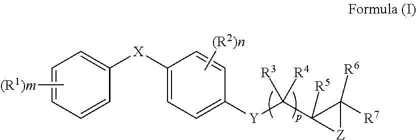

wherein:

m is 0, 1, 2, 3, 4 or 5;

n is 0, 1, 2, 3, 4 or 5;

p is 1, 2 or 3;

X is —O—, —S—, —CH₂— or a direct bond;

Y is —C(O)— or —S(O)₂—,

Z is —O— or —S—;

R¹ at each occurrence is the same or different and independently alkyl, alkenyl, aralkyl, haloalkyl, halogen, —OR⁸ or —NR⁹R¹⁰;

R² at each occurrence is the same or different and independently alkyl, alkenyl, aralkyl, haloalkyl, halogen, —OR⁸ or —NR⁹R¹⁰;

R³ and R⁴ are each the same or different and independently hydrogen or alkyl;

R⁵, R⁶ and R⁷ are each the same or different and independently hydrogen or alkyl;

R⁸ is hydrogen, alkyl, alkenyl, or aryl;

R⁹ and R¹⁹ are each the same or different and independently hydrogen or alkyl. Pharmaceutically acceptable salts of the compounds described herein are also contemplated.

### DEFINITIONS

“Alkyl” refers to a straight or branched hydrocarbon chain radical consisting solely of carbon and hydrogen atoms, containing no unsaturation, having from one to fifteen carbon atoms. In certain embodiments, an alkyl may comprise one to eight carbon atoms. In other embodiments, an alkyl may comprise one to six carbon atoms. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one or more of the following substituents: halo, cyano, nitro, oxo, thioxo, trimethylsilanyl, —OR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, haloalkyl, aryl or aralkyl.

“Alkenyl” refers to a straight or branched hydrocarbon chain radical group consisting solely of carbon and hydrogen atoms, containing at least one double bond, and having from two to twelve carbon atoms. In certain embodiments, an alkenyl may comprise two to eight carbon atoms. In other embodiments, an alkenyl may comprise two to four carbon atoms. The alkenyl is to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group may be optionally substituted by one or more of the following substituents: halo, cyano, nitro, oxo, thioxo, trimethylsilanyl, —OR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, haloalkyl, aryl or aralkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl, fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, haloalkyl, cyano, nitro, optionally substituted aryl, optionally substituted aralkyl.

“Aralkyl” refers to a radical of the formula —R^{b}-aryl where R^{b} is an alkylene chain, which refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Exemplary aralkyls include benzyl, diphenylmethyl and the like. The alkylene chain part of the aralkyl radical may be optionally substituted as described above for an alkyl. The aryl part of the aralkyl radical may be optionally substituted as described above for an aryl group.

“Halogen” refers to bromo, chloro, fluoro or iodo.

“Haloalkyl” refers to an alkyl radical, as defined above, that is substituted by one or more halo radicals, as defined above, for example, trifluoromethyl, difluoromethyl, 2,2,2-trifluoroethyl, 1-fluoromethyl-2-fluoroethyl, trichloromethyl and the like. The alkyl part of the haloalkyl radical may be optionally substituted as defined above for an alkyl group.

### Sub-Genuses of Formula (I):

In certain embodiments, X is —O—, Y is —S(O)₂— and Z is —S—; and compounds of Formula (I) can be represented by Formula (Ia):

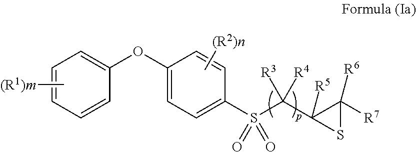

In further embodiments of Formula (Ia), m is 0, n is 0, p is 1, R³, R⁴, R⁵, R⁶ and R⁷ are each hydrogen, and Formula (Ia) is SB-3CT:

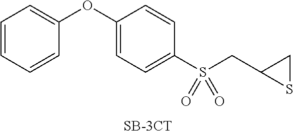

In certain other embodiments, X is —S—, Y is —S(O)₂— and Z is —S—; and compounds of Formula (I) can be represented by Formula (Ib):

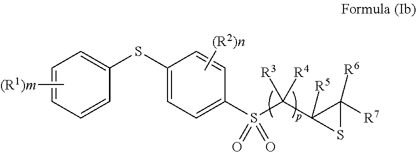

In certain other embodiments, X is —CH₂—, Y is —S(O)₂— and Z is —S—; and compounds of Formula (I) can be represented by Formula (Ic):

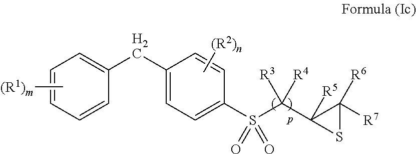

### Method of Making Compounds of Formula (I), (Ia)-(Ic):

Compounds of Formula (I), including those of Formula (Ia)-(Ic), can be prepared according to the following general reaction scheme:

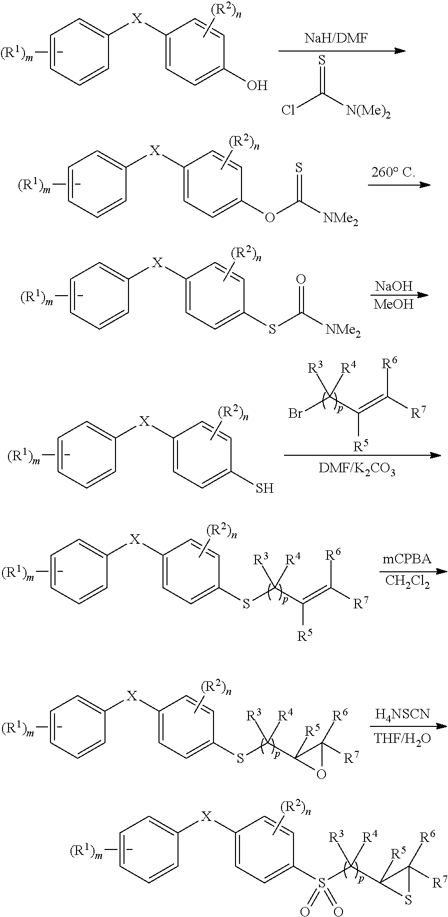

### Other MMP2 and MMP 9 Inhibitors

Compounds or agents of the present invention that inhibit MMP2 and/or MMP9 activity, either gene expression or activity of the protein, may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or may be readily produced. Additionally, natural or synthetically produced libraries and compounds can be readily modified through conventional chemical, physical and biochemical means, and may be used to produce combinatorial libraries. Known pharmacological agents may also be subjected to directed or random chemical modifications, such as acylation, alkylation, esterification, amidification, etc. to produce structural analogs. New potential therapeutic agents may also be created using methods such as rational drug design or computer modeling.

Agents for use in inhibiting MMP2 and/or MMP9 according to the present invention may be screened from “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be contacted with or administered to purified MMP2 and/or MMP9 or administered in vivo in an appropriate animal model, such as a murine or rat model such as described herein. Compounds so identified as capable of inhibiting MMP2 and/or MMP9 may be valuable for therapeutic purposes, since they permit treatment of diseases as described herein and for therapeutic use as immunosuppressive agents.

Agents that inhibit MMP2 and/or MMP9 further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629). Those having ordinary skill in the art will appreciate that a diverse assortment of such libraries may be prepared according to established procedures, and tested using screening methods known in the art.

Agents and compounds that inhibit MMP2 and/or MMP9 of the present invention may also include antibodies that bind to the MMP2 and/or MMP9 polypeptide. Antibodies may function as modulating agents to inhibit or block activity of the polypeptides of the present invention in vivo. Alternatively, or in addition, antibodies may be used within screens for endogenous activity of MMP2 and/or MMP9, or as modulating agents, for purification of said polypeptides, for assaying the level of activity of said polypeptides within a sample and/or for studies of expression of said polypeptides. Such antibodies may be polyclonal or monoclonal, and are generally specific for MMP2 and/or MMP9. Within certain embodiments, antibodies are polyclonal.

Antibodies may be prepared by any of a variety of techniques known to those of ordinary skill in the art (see, e.g., Harlow and Lane, Antibodies: A Laboratory Manual, Cold Spring Harbor Laboratory, 1988). In one such technique, an immunogen comprising an SPL polypeptide or antigenic portion thereof is initially injected into a suitable animal (e.g., mice, rats, rabbits, sheep and goats), preferably according to a predetermined schedule incorporating one or more booster immunizations. The use of rabbits is preferred. To increase immunogenicity, an immunogen may be linked to, for example, glutaraldehyde or keyhole limpet hemocyanin (KLH). Following injection, the animals are bled periodically to obtain post-immune serum containing polyclonal antibodies that bind to MMP2 and/or MMP9. Polyclonal antibodies may then be purified from such antisera by, for example, affinity chromatography using an MMP2 and/or MMP9 polypeptide, or antigenic portion thereof coupled to a suitable solid support. Such polyclonal antibodies may be used directly for screening purposes and for Western blots.

More specifically, an adult rabbit (e.g., NZW) may be immunized with 10 μg purified (e.g., using a nickel-column) SK or SPL polypeptide emulsified in complete Freund's adjuvant (1:1 v/v) in a volume of 1 mL. Immunization may be achieved via injection in at least six different subcutaneous sites. For subsequent immunizations, 5 μg of an MMP2 or MMP9 polypeptide may be emulsified in complete Freund's adjuvant and injected in the same manner. Immunizations may continue until a suitable serum antibody titer is achieved (typically a total of about three immunizations). The rabbit may be bled immediately before immunization to obtain pre-immune serum, and then 7-10 days following each immunization.

For certain embodiments, monoclonal antibodies may be desired. Monoclonal antibodies may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

An antibody that specifically binds to MMP2 and/or MMP9 may interact with said polypeptide via specific binding if the antibody binds the polypeptide with a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹ to 10⁹ M⁻¹. Affinities of binding partners such as antibodies and the polypeptides that they bind to can be readily determined using conventional techniques, for example those described by Scatchard et al., Ann. N.Y. Acad. Sci. 51:660 (1949) and in Current Protocols in Immunology, or Current Protocols in Cell Biology, both published by John Wiley & Sons, Inc., Boston, Mass.

As noted above, the present invention provides agents or compounds that alter the expression (transcription or translation), stability and/or activity of an MMP2 and/or MMP9 polypeptide. To identify such a modulating agent, any of a variety of screens may be performed. Candidate modulating agents may be obtained using well known techniques from a variety of sources, such as plants, fungi or libraries of chemicals, small molecules or random peptides. Antibodies that bind to an MMP2 or MMP9 polypeptide of the present invention, and anti-sense polynucleotides that hybridize to a polynucleotides that encodes an MMP2 and/or MMP9 protein may be used in the methods of the invention for inhibiting MMP2 and MMP9 and may function as immunosuppressive agents. In certain embodiments, such inhibitor agents have a minimum of side effects and are non-toxic. For some applications, agents that can penetrate cells are preferred.

Agents that inhibit MMP2 and/or MMP9 encompass numerous chemical classes, though typically they are organic molecules, preferably small organic compounds having a molecular weight of more than 50 and less than about 2,500 daltons. Inhibitory agents comprise functional groups necessary for structural interaction with proteins, particularly hydrogen bonding, and typically include at least an amine, carbonyl, hydroxyl or carboxyl group, preferably at least two of the functional chemical groups. The inhibitory agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Agents that inhibit MMP2 and/or MMP9 activity are described herein and additional suitable agents for use according to the present embodiments may be identified according to routine methodologies, such as those described in the herein incorporated references. For instance, methods of detecting MMP2 and/or MMP9 activity are described herein in the examples. Methods of screening compound libraries for agents that inhibit MMP2 and MMP9 activity, including polynucleotide sequences for the production of nucleic acid molecules that encode MMP polypeptides and the production of MMP polypeptides therefrom, are known in the art and are commercially available. See for example, R&D Systems, Minneapolis, Minn.; Calbiochem® (EMD/Merck, Darmstadt, Germany). For embodiments that relate to molecular biology methodologies, compositions and methods well known to those of ordinary skill in the art are described for example, in Sambrook et al., Molecular Cloning: A Laboratory Manual, Cold Spring Harbor Laboratories, Cold Spring Harbor, N.Y., 1989; Ausubel et al. (1993 Current Protocols in Molecular Biology, Greene Publ. Assoc. Inc. & John Wiley & Sons, Inc., Boston, Mass.); Maniatis et al. (1982 Molecular Cloning, Cold Spring Harbor Laboratory, Plainview, N.Y.) and elsewhere. Certain embodiments as provided herein expressly contemplate a method of modulating immune function in a subject that comprises administering an agent that inhibits MMP2 and/or MMP9 such as SB-3CT, optionally in combination with one or more additional agents, such as other immunosuppressive agents.

As also provided herein, certain contemplated embodiments relate to a method of inhibiting immune function in a subject by administering an agent that decreases MMP2 and/or MMP9 activity, which in certain embodiments may involve an agent that decreases MMP2 and/or MMP9 activity by directly binding to the proteins, while in certain other embodiments an agent that decreases MMP2 and/or MMP9 activity may do so indirectly, for example, by interacting with other cellular molecular components that exert an effect on MMP activity. Certain contemplated embodiments relate to an agent that is capable of decreasing MMP2 and/or MMP9 activity by causing a decreased expression level of either protein. Abundant disclosure describing nucleic acid molecules that encode MMP2 and/or MMP9 polypeptides and how to measure them may be found in the public databases including GENBANK™ and SWISSPROT™, and PubMed. See also, Cancer Res. 68 (21), 9096-9104 (2008), Biomed Khim. 2008 September-October; 54(5):555-60; Cancer Invest. 2008 December; 26(10):984-9; Oncol Rep. 2002 May-June; 9(3):607-11. As would be readily appreciated by the skilled person, nucleotides that hybridize to the polynucleotides encoding MMP2 and/or MMP9 are contemplated herein such as nucleotides that hybridize under moderately stringent conditions, which may be, e.g., prewashing in a solution of 5×SSC, 0.5% SDS, 1.0 mM EDTA (pH 8.0); hybridizing at 50-65° C., 5×SSC, overnight; followed by washing twice at 65° C. for 20 minutes with each of 2×, 0.5× and 0.2×SSC containing 0.1% SDS).

According to certain related embodiments, an agent that causes a decreased MMP2 and/or MMP9 expression level may be an antisense polynucleotide that specifically hybridizes to a nucleic acid molecule that encodes an MMP2 and/or MMP9 polypeptide, a ribozyme that specifically cleaves a nucleic acid molecule that encodes an MMP2 or MMP9 polypeptide, a small interfering RNA that is capable of interfering with a nucleic acid molecule that encodes an MMP2 and/or MMP9 polypeptide, or an agent that alters activity of a regulatory element that is operably linked to a nucleic acid molecule that encodes an MMP2 and/or MMP9 polypeptide. As disclosed herein and known to the art, such nucleic acid sequence-based agents can be readily prepared using routine methodologies.

A polynucleotide that is complementary to at least a portion of a coding sequence (e.g., an antisense polynucleotide, siRNA or a ribozyme) may thus be used to modulate MMP2 and/or MMP9-encoding gene expression. Identification of oligonucleotides, siRNA and ribozymes for use as antisense agents, and DNA encoding genes for their targeted delivery, involve methods well known in the art. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrahedron Lett. 28:3539-3542 (1987); Miller et al., J. Am. Chem. Soc. 93:6657-6665 (1971); Stec et al., Tetrahedron Lett. 26:2191-2194 (1985); Moody et al., Nucl. Acids Res. 12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)).

Antisense polynucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary to or bind the sense strand of DNA or mRNA that encodes an MMP2 and/or MMP9 polypeptide or a protein mediating any other process related to expression of endogenous MMP2 and/or MMP9, such that inhibition of translation of mRNA encoding the MMP2 and/or MMP9 polypeptide is affected. cDNA constructs that can be transcribed into antisense RNA may also be introduced into cells or tissues to facilitate the production of antisense RNA. Antisense technology can be used to control gene expression through interference with binding of polymerases, transcription factors or other regulatory molecules (see Gee et al., In Huber and Carr, Molecular and Immunologic Approaches, Futura Publishing Co. (Mt. Kisco, N.Y.; 1994)). Alternatively, an antisense molecule may be designed to hybridize with a control region of a MMP-encoding gene (e.g., promoter, enhancer or transcription initiation site), and block transcription of the gene; or to block translation by inhibiting binding of a transcript to ribosomes.

The present invention also contemplates use of MMP2 and/or MMP9-encoding nucleic acid sequence-specific ribozymes. A ribozyme is an RNA molecule that specifically cleaves RNA substrates, such as mRNA, resulting in specific inhibition or interference with cellular gene expression. There are at least five known classes of ribozymes involved in the cleavage and/or ligation of RNA chains. Ribozymes can be specifically targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). Any MMP2 and/or MMP9 mRNA-specific ribozyme, or a nucleic acid encoding such a ribozyme, may be delivered to a host cell to effect inhibition of MMP2 and/or MMP9 gene expression. Ribozymes may therefore be delivered to the host cells by DNA encoding the ribozyme linked to a eukaryotic promoter, such as a eukaryotic viral promoter, such that upon introduction into the nucleus, the ribozyme will be directly transcribed. Particularly useful sequence regions of a MMP2 and/or MMP9-encoding mRNA for use as a ribozyme target can be found using routine sequence alignment tools known to the art such as BLAST or MegAlign, and may preferably be sequence stretches that are unique to the MMP2 and/or MMP9-encoding mRNA relative to other transcribed sequences that may be present in a particular cell.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine and wybutosine, as well as acetyl-methyl-, thio- and other modified forms of adenine, cytidine, guanine, thymine and uridine.

RNA interference (RNAi) is a polynucleotide sequence-specific, post-transcriptional gene silencing mechanism effected by double-stranded RNA that results in degradation of a specific messenger RNA (mRNA), thereby reducing the expression of a desired target polypeptide encoded by the mRNA (see, e.g., WO 99/32619; WO 01/75164; U.S. Pat. No. 6,506,559; Fire et al., Nature 391:806-11 (1998); Sharp, Genes Dev. 13:139-41 (1999); Elbashir et al. Nature 411:494-98 (2001); Harborth et al., J. Cell Sci. 114:4557-65 (2001)). “Small interfering RNA” (siRNA) or DNP-RNA polynucleotides that interfere with expression of specific polypeptides in higher eukaryotes such as mammals (including humans) have been considered (e.g., Karagiannis and El-Osta, 2005 Cancer Gene Ther. May 2005, PMID: 15891770; Chen et al., 2005 Drug Discov. Today 10:587; Scherr et al., 2005 Curr. Opin. Drug Discov. Devel. 8:262; Tomari and Zamore, 2005 Genes Dev. 19:517; see also, e.g., Tuschl, 2001 Chembiochem. 2:239-245; Sharp, 2001 Genes Dev. 15:485; Bernstein et al., 2001 RNA 7:1509; Zamore, 2002 Science 296:1265; Plasterk, 2002 Science 296:1263; Zamore 2001 Nat. Struct. Biol. 8:746; Matzke et al., 2001 Science 293:1080; Scadden et al., 2001 EMBO Rep. 2:1107; Hutvagner et al., Curr. Opin. Gen. Dev. 12:225-32 (2002); Elbashir et al., 2001; Nykänen et al., Cell 107:309-21 (2001); Bass, Cell 101:235-38 (2000)); Zamore et al., Cell 101:25-33 (2000)). Transfection of human and other mammalian cells with double-stranded RNAs of about 18-27 nucleotide base pairs in length interferes in a sequence-specific manner with expression of particular polypeptides encoded by messenger RNAs (mRNA) containing corresponding nucleotide sequences (WO 01/75164; Elbashir et al., 2001; Elbashir et al., Genes Dev. 15:188-200 (2001)); Harborth et al., J. Cell Sci. 114:4557-65 (2001); Carthew et al., Curr. Opin. Cell Biol. 13:244-48 (2001); Mailand et al., Nature Cell Biol. Advance Online Publication (Mar. 18, 2002); Mailand et al. 2002 Nature Cell Biol. 4:317).

As noted above, in certain embodiments the agent that causes a decreased MMP2 and/or MMP9 expression level may alter activity of a regulatory element that is operably linked to a nucleic acid molecule that encodes an MMP2 and/or MMP9 polypeptide. By way of representative example and not limitation, these and related embodiments contemplate suitable agents that are capable of down-regulating MMP2 and/or MMP9 activity by suppressing or repressing transcription of MMP2 and/or MMP9-encoding genes, which agents can be readily identified using art-accepted methodologies to screen for functional blockers of MMP2 and/or MMP9 gene transcription.

### Methods of Use

The methods of the present invention may be used in the context of a variety of disease settings where inhibiting an immune response may be desired. The present invention centers on the unexpected discovery that MMP2 and MMP9 are present intracellularly and regulate T cell activation. Thus, the present invention provides methods for inhibiting immune responses by targeted inhibition of MMP2 and MMP9. In particular, the present invention provides methods for inhibiting an immune response in a patient or subject in need thereof by specifically inhibiting MMP2 and/or MMP9 by administering to the patient a therapeutically effective amount of an MMP2- and/or MMP9-specific inhibitor, such as the compounds described herein. In this regard, the present invention may be used to inhibit the immune response in any of a variety of autoimmune diseases, including but not limited to, alloimmune-induced autoimmunity post organ transplant (heart, lung, liver, kidney, pancreas, multi-visceral transplant, hematopoetic stem cell); collagen vascular diseases (systemic lupus erythematosus, rheumatoid arthritis, Wegener's granulomatosis, scleroderma), rheumatoid arthritis, multiple sclerosis, insulin dependent diabetes, Addison's disease, celiac disease, chronic fatigue syndrome, inflammatory bowel disease, ulcerative colitis, Crohn's disease, Fibromyalgia, systemic lupus erythematosus, psoriasis, Sjogren's syndrome, hyperthyroidism/Graves disease, hypothyroidism/Hashimoto's disease, Insulin-dependent diabetes (type 1), Myasthenia Gravis, endometriosis, scleroderma, pernicious anemia, Goodpasture syndrome, Wegener's disease, glomerulonephritis, aplastic anemia, paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever.

The methods provided herein are also contemplated for reducing an immune response in such disease settings as asthma, idiopathic pulmonary fibrosis, fibrotic disorders in organs, injuries such as ventilator-induced lung injury, ischemia reperfusion injury, ozone lung injury, spinal cord injury, chronic obstructive pulmonary disease (COPD), Steven's Johnson syndrome, and herpes simplex virus encephalitis.

The present invention provides methods for reducing alloantigen induced T cells proliferation in solid organ transplant settings. In this regard, the methods of the invention may be used in the context of any solid organ transplant, including, but not limited to, lung, heart, kidney, liver, pancreas, and intestine transplants. Thus the present invention provides methods for reducing alloantigen-induced proliferation of T cells comprising, administering to a transplant patient a therapeutically effective amount of an MMP2- and/or MMP9-specific inhibitor. In certain embodiments of the invention, the inhibitor comprises a compound of Formula I or other related compound as described herein, or an siRNA molecule that down regulates expression of a MMP2 and/or MMP9, or an antibody that blocks the activity of MMP2 and/or MMP9. In certain embodiments, the present invention provides for administering prior to organ harvest, a therapeutically effective amount of an MMP2 and/or MMP9-specific inhibitor, such as those described herein, to an organ donor donating an organ to the transplant patient. This further reduces the alloantigen-induced response.

In a further embodiment, the present invention provides methods for inhibiting an immune response against a collagen in a transplant patient or a patient in need of a transplant comprising administering to the patient an effective amount of a specific inhibitor of MMP2 and/or MMP9. In certain embodiments, the transplant patient is a lung transplant recipient. In a related embodiment of the invention, in certain settings, it may be desirable to administer a specific inhibitor of MMP2 and/or MMP9 in conjunction with administration of collagen V, either orally, by i.v. or by other routes described herein.

The present invention also provides methods for improving the outcome of a transplant comprising, administering to a transplant patient a therapeutically effective amount of an MMP2- and/or MMP9-specific inhibitor, such as the compounds described herein. In certain embodiments, it may be desirably to administer prior to organ harvest, a therapeutically effective amount of an MMP2 and/or MMP9 inhibitor, such as the compounds described herein, to an organ donor donating an organ to the transplant patient. By “improving the outcome” is meant improving acceptance of graft, reducing graft rejection or graft versus host disease, and preservation of oxygenation of the graft post transplantation.

Immunosuppressive drugs are well known to be highly toxic. Steroidal drugs have been used for decades and their adverse effects are well known. Adverse effects that can be anticipated in all patients on prolonged steroid therapy include osteoporosis, truncal obesity, impaired wound healing, infections and growth arrest in children. Less frequently occurring adverse effects include myopathy, hypertension, hyperlipidemia, diabetes mellitus and cataracts. Severe side effects may develop and require patient monitoring. These include glaucoma, intracranial hypertension, intestinal perforation, and ulcers.

If autoimmune diseases such as myasthenia gravis (MG), rheumatoid arthritis (RA) systemic lupus erythematosus (SLE), multiple sclerosis (MS) and juvenile arthritis, often treated first with corticosteroids, become refractory to steroids, then increasingly toxic drugs are employed, including azathioprine, methotrexate and cyclophosphamide. The primary effect of azathioprine is inhibiting DNA synthesis, thus lowering numbers of T and B lymphocytes. In addition, azathioprine inhibits the mixed lymphocyte reaction and immunoglobulin production, but does not consistently affect delayed-type hypersensitivity. The major adverse effect of azathioprine is pancytopenia, particularly lymphopenia and granulocytopenia. Consequently, there are increased risks of viral, fungal, mycobacterial and protozoal infections. An increased rate of lymphoreticular malignancies has been reported in kidney transplant patients, but not in patients with RA.

Methotrexate inhibits folic acid synthesis and is cytotoxic, suppressing bone marrow. At the low doses used for RA, methotrexate should not decrease the numbers of lymphocytes; but IgM and IgG are reduced. Side effects include pneumonia, nausea, stomach upsets, mouth ulcers, leukopenia, throubocytopenia, and a form of hepatic fibrosis, which can only be diagnosed by liver biopsy.

Cyclophosphamide is also used in RA therapy. It is metabolized in the liver to a compound which cross-links DNA. Cyclophosphamide is cytotoxic, with severe toxicity seen even at low doses. It affects RA by reducing numbers of B- and T-lymphocytes, decreasing the immunoglobulin concentrations and diminishing B-cell responsiveness to mitogenic stimuli. Hair loss, infections, and powerful nausea are common. With prolonged administration, patients develop malignancies at an increased rate.

Cyclosporin does not suppress white cells, but it is a powerful immunomodulatory drug and is effective in treating rheumatoid arthritis. However, an important side effect is renal toxicity.

Monoclonal antibodies to CD4 have been used in autoimmune diseases, but they cause nonspecific immunosuppression. It has been recommended that new therapies interfere with the initial presentation of specific inciting antigens to T-lymphocytes. (Wraith et al., Cell (1989) 57:709-715).

Other drugs have been used specifically in RA, including gold salts, antimalarials, sulfasalazine and penicillamine. Gold salts are given intramuscularly and their effect may not be seen for months. Adverse effects of gold treatment include bone marrow aplasia, glomerulonephritis, pulmonary toxicity, vasomotor reactions and inflammatory flare. Antimalarials exert several effects on the immune system without decreasing the numbers of lymphocytes. The most serious side effects of antimalarials include retinal pigment deposition, rash and gastrointestinal upset. Sulfasalazine has several effects which contribute to its effect on RA; however, it has numerous side effects. Penicillamine has been successfully used in RA; however, its numerous side effects have limited its use. Penicillamine has been reported to cause other autoimmune diseases, including myasthenia gravis and SLE.

When patients receive allografts (transplanted tissue from other humans or other sources), their immune systems can destroy the allografts quickly absent the administration of immunosuppressant drugs. A number of different organs and tissues are now transplanted, including the kidneys, heart, lungs, skin, bone marrow, cornea, and liver. Drugs frequently used in transplant patients include cyclosporin, azathioprine, rapamycin, other macrolides such as FK506, prednisone, methylprednisolone, CD4 antibodies and cyclophosphamide. Frequently these drugs must be given in higher doses and for longer periods to transplant patients than to patients with autoimmune diseases. Hence, side effects from these drugs (discussed above) may be more common and severe in transplant patients.

In summary, immunosuppressive drugs are well known to be highly toxic. Reducing the dosage needed by combining treatment with MMP2 and/or MMP9 inhibitors would be advantageous. Thus, the present invention further provides methods for reducing the dose of toxic immunosuppressants necessary by combining administration of an inhibitor specific for MMP2 and/or MMP9 with the administration of any of a variety of known immunosuppressive drugs, such as cyclosporin, tacrolimus (FK506), sirolumus (rapamycin), methotrexate, azathioprine, mercaptopurine, cytotoxic antibiotics, such as dactinomycin, mitomycin C, bleomycin, and mithramycin, cyclophosphamide, purine analogs, glucocorticoids, antibodies (e.g., anti-CD20, anti-CD3 and anti-L-2 receptor), interferons, TNF binding proteins, and mycophenolate.

The present invention also provides methods for reducing or inhibiting an immune response by administering a specific inhibitor of MMP2 and/or MMP9 in combination with other known therapies, including other immunosuppressive drugs.

“Immune response” as used herein, refers to activation of cells of the immune system, including but not limited to, T cells, B cells, macrophages, and dendritic cells, such that a particular effector function(s) of a particular cell is induced. Effector functions may include, but are not limited to, presentation of antigen, proliferation, secretion of cytokines, secretion of antibodies, expression of regulatory and/or adhesion molecules, expression of activation molecules, and the ability to induce cytolysis. Any T cell of the immune system may be part of the “immune response” as used herein, such as CD8+ T cells, CD4+ T cells, regulatory T cells, allo-reactive T cells, antigen-specific T cells, memory T cells. As would be recognized by the skilled person, cells of the immune system can be identified, purified, or otherwise measured by expression patterns of cell surface markers, cytokine expression patterns or effector function.

As used herein, “reducing or inhibiting an immune response” means decreasing either the amount of a component of the immune system (e.g., a cytokine) or the activity by which a component of the immune system is characterized. By way of example, inhibiting an immune response of a subject includes increasing the number of suppressor or regulatory T lymphocytes present, increasing secretion of immunosuppressive factors by a suppressor or regulatory T lymphocyte in the subject, decreasing the number of cytotoxic T lymphocytes present in the subject, decreasing the cytotoxic activity of a cytotoxic T lymphocyte in the subject, decreasing the amount of an antibody, decreasing the amount of a complement protein, decreasing the ability of a complement protein to interact with a cell, and the like. Therefore, “reducing” or “inhibiting” may mean an increase in the activity or amount of certain immunomodulatory cytokines or certain cells of the immune system, such as regulatory T cells.

Assays and methods for measuring changes in immune responses are well known in the art. For example, components of the immune system can be measured systemically (e.g., from peripheral blood) or locally (e.g., from specific cell samples such as spleen cells, lymph node cells, tumors, MALT, GALT, etc.) by measuring the levels of a variety of cytokines, using any of a number of assays known in the art, such as those described in Current Protocols in Immunology, Edited by: John E. Coligan, Ada M. Kruisbeek, David H. Margulies, Ethan M. Shevach, Warren Strober (2001 John Wiley & Sons, NY, N.Y.). A variety of protocols for detecting and measuring the expression of cytokines, using either polyclonal or monoclonal antibodies specific for the cytokine are known in the art. Examples include enzyme-linked immunosorbent assay (ELISA), ELISPOT, intracellular cytokine staining assay (ICS,) radioimmunoassay (RIA), fluorescence activated cell sorting (FACS), and cell-based assays such as IL-2 dependent T cell assay. A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A variety of cell assays to measure increases and decreases in effector function of the immune response are well known to the skilled person and are described, for example, in Current Protocols in Immunology, Edited by: John E. Coligan, Ada M. Kruisbeek, David H. Margulies, Ethan M. Shevach, Warren Strober (2001 John Wiley & Sons, NY, N.Y.). These include proliferation assays, cytotoxic T cell assays (e.g., chromium release or similar assays), intracellular cytokine staining assays, ELISPOT, and gene expression analysis using any number of polymerase chain reaction (PCR) or RT-PCR based assays. General assays and techniques that may be useful for practicing the methods described herein may also be found in, for example, Methods Ausubel et al. (2001 Current Protocols in Molecular Biology, Greene Publ. Assoc. Inc. & John Wiley & Sons, Inc., NY, N.Y.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.) and elsewhere. Measurements of antibody production, either specific antibodies or antibodies generally, can also be used to measure an immune response and changes thereto.

Generally, reducing or inhibiting an immune response comprises a decrease in a humoral response and/or a cellular response but as noted elsewhere herein, may comprise an increase in the number and/or activity of regulatory or suppressor T cells and/or cytokines produced by such cells. As such “inhibition” or “reduction” of an immune response comprises any statistically significant decrease (or increase where appropriate, such as in regulatory or suppressor T cells), in the level of one or more appropriate immune cells (T cells, B cells, antigen-presenting cells, dendritic cells, and the like) or in the activity of one or more of these immune cells (CTL activity, helper T lymphocyte (HTL) activity), cytokine secretion, change in profile of cytokine secretion, etc.), as measured using techniques known in the art and described herein.

In certain embodiments, inhibition of an immune response comprises a decrease in antigen-specific or alloreactive T cell activity of between 1.5 and 5 fold in a subject administered an MMP2 and/or MMP9 inhibitor. In another embodiment, inhibition of an immune response comprises a decrease in antigen-specific or alloreactive T cell activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered an MMP2 and/or MMP9 inhibitor as described herein.

In a further embodiment, inhibition of an immune response comprises a decrease in antigen-specific or alloreactive HTL activity, such as proliferation of helper T cells, of between 1.5 and 5 fold in a subject administered an MMP2 and/or MMP9 inhibitor as described herein. In another embodiment, inhibition of an immune response comprises a decrease in antigen-specific or alloreactive HTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered an MMP2 and/or MMP9 inhibitor as described herein. In this regard, inhibition in HTL activity may comprise a decrease in production of one or more of particular cytokines, such as interferon-gamma (IFN-γ), interleukin-1 (IL-1), IL-2, IL-3, IL-6, IL-7, IL-12, IL-15, tumor necrosis factor-alpha (TNF-α), granulocyte macrophage colony-stimulating factor (GM-CSF), granulocyte-colony stimulating factor (G-CSF), or other cytokines.

In a further embodiment, inhibition of an immune response comprises a decrease in antigen-specific or alloreactive CTL activity, such as proliferation of cytotoxic T cells, of between 1.5 and 5 fold in a subject administered an MMP2 and/or MMP9 inhibitor as described herein. In another embodiment, inhibition of an immune response comprises a decrease in antigen-specific or alloreactive CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered an MMP2 and/or MMP9 inhibitor as described herein. In this regard, inhibition in CTL activity may comprise a decrease in cytotoxic activity of CD8+ T cells as measured by an appropriate assay known in the art (e.g., Chromium release assay; intracellular cytokine staining assay, ELISPOT).

In a further embodiment, reducing or inhibiting of an immune response comprises a decrease in specific antibody production of between 1.5 and 5 fold in a subject administered the MMP2 and/or MMP9 inhibitors by the methods of the present invention. In another embodiment, reducing or inhibiting of an immune response comprises a decrease in specific antibody production of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the MMP2 and/or MMP9 inhibitors by the methods of the present invention.

In certain embodiments of the invention, administration of the MMP2 and/or MMP9 inhibitors of the invention do not affect regulatory T cells. Regulatory T cells can be measured using the assays as described herein and may be identified by cell surface marker expression. In particular, as would be understood by the skilled artisan, classically, T regulatory cells have a CD4⁺, CD25⁺, CD62L^{hi}, GITR⁺, and FoxP3⁺ phenotype (see for example, Woo, et al., J. Immunol. 2002 May 1; 168(9):4272-6; Shevach, E. M., Annu. Rev. Immunol. 2000, 18:423; Stephens, et al., Eur. J. Immunol. 2001, 31:1247; Salomon, et al, Immunity 2000, 12:431; and Sakaguchi, et al., Immunol. Rev. 2001, 182:18). Other markers may also be useful in the identification and quantification of regulatory T cells (see e.g., Inflamm Allergy Drug Targets. 2008 December; 7(4):217-23).

Subject as used herein refers to any mammal. In certain embodiments, the subject is human patient. In further embodiments, the subject may be a mouse, rat, dog, cat, non-human primate, pig or other laboratory animal. In certain embodiments, the subject is a human patient in need of immunosuppressive therapy, a patient in need of a transplant or a transplant patient.

As would be readily appreciated by the skilled artisan, other measures can be used to measure inhibition or reduction of an immune response such as clinical indications of an immune response including, but not limited to, reduction or improvement in transplant rejection, reduction in GVHD or host versus graft disease, reduction in autoimmune symptoms and the like.

### Pharmaceutical Compositions

Administration of the MMP2 and MMP9 inhibitor compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other immunosuppressive agents) and/or suitable excipients such as salts, buffers and stabilizers may, but need not, be present within the composition.

Administration may be achieved by a variety of different routes, including oral, parenteral, nasal, intravenous, intradermal, subcutaneous or topical. Preferred modes of administration depend upon the nature of the condition to be treated or prevented. An amount that, following administration, reduces, inhibits, prevents or delays the onset of an immune response or clinical indication of such a response is considered effective.

In certain embodiments, the amount administered is sufficient to result in reduced immune activity as described elsewhere herein. The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The compounds of the present invention may be administered alone or in combination with other known treatments, such as immunosuppressive regimens, radiation therapy, chemotherapy, transplantation, oral collagen therapy, immunotherapy, hormone therapy, photodynamic therapy, etc.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Certain oral pharmaceutical compositions contain between about 4% and about 75% of the compound of the invention. Certain pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

Compounds of the invention, or pharmaceutically acceptable salts thereof, may also be administered simultaneously with, prior to, or after administration of one or more other therapeutic agents. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

The compounds of the present invention may be administered to an individual afflicted with a disease or disorder as described herein, such as an autoimmune disease or disorders associated with organ transplantation. For in vivo use for the treatment of human disease, the compounds described herein are generally incorporated into a pharmaceutical composition prior to administration. A pharmaceutical composition comprises one or more of the compounds described herein in combination with a physiologically acceptable carrier or excipient as described elsewhere herein. To prepare a pharmaceutical composition, an effective amount of one or more of the compounds is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

The compounds described herein may be prepared with carriers that protect it against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

### EXAMPLES

### Example 1

### MMP9 is Expressed in CD4⁺ and CD8⁺ T Cells

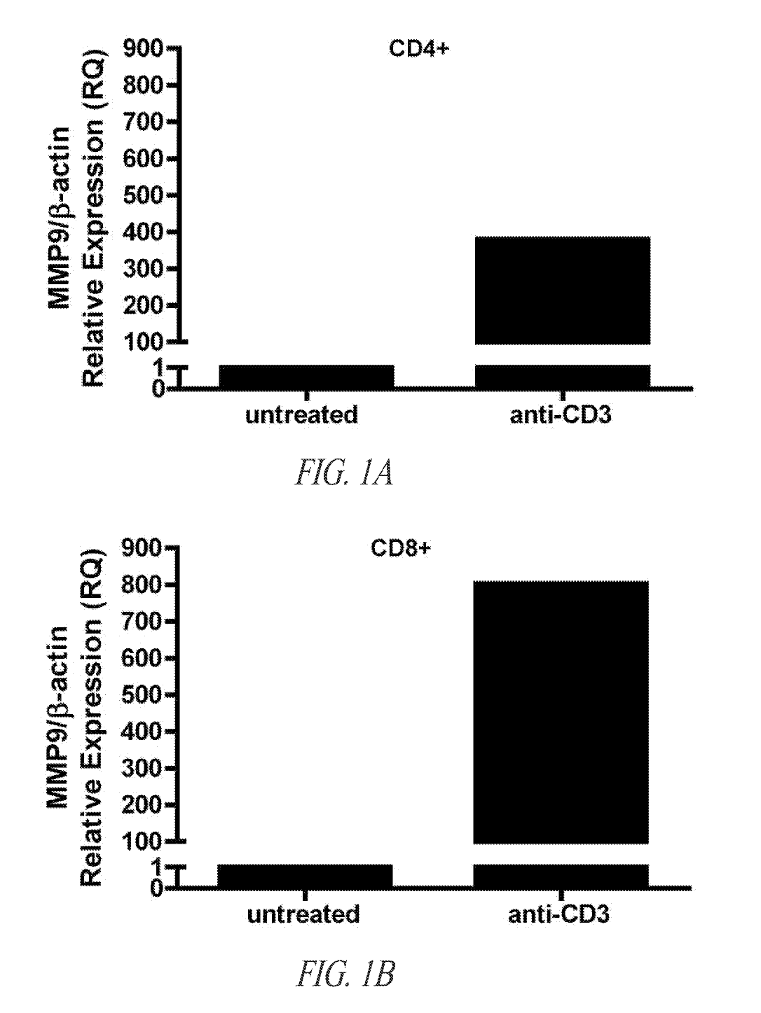

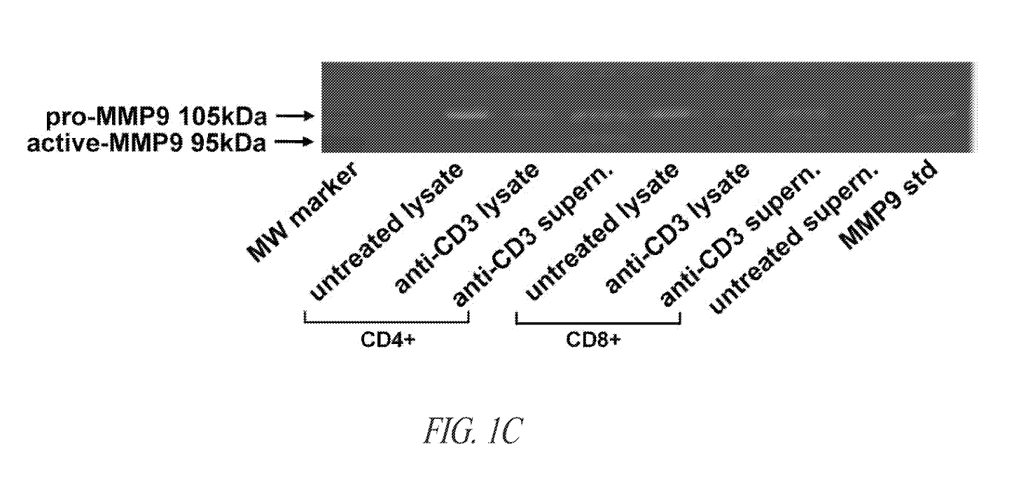

To begin to address the role of MMPs in T cell activation, the mRNA and protein expression pattern of MMP9 was measured in cell lysates and conditioned media of activated murine splenic CD4⁺ and CD8⁺ T cells by means of quantitative RT PCR and substrate zymography, respectively. As shown in FIG. 1, there were detectable levels of MMP9 mRNA expression in unstimulated CD4⁺ (FIG. 1A) and CD8⁺ (FIG. 1B) T cells. Following anti-CD3 antibody stimulation, MMP9 mRNA transcript levels were increased in both cell populations although CD8⁺ transcript levels were more pronounced. Analysis of MMP9 protein expression (FIG. 1C) revealed increased expression of pro-MMP9 in untreated CD4⁺ and CD8⁺ T cell lysates. Following stimulation with anti-CD3 antibody, pro-MMP9 expression is slightly diminished in the T cell lysates and active MMP9 is expressed in the supernatant.

### Example 2

### Broad-Spectrum MMP Inhibition Abrogates Anti-CD3-Induced T Cell Proliferation

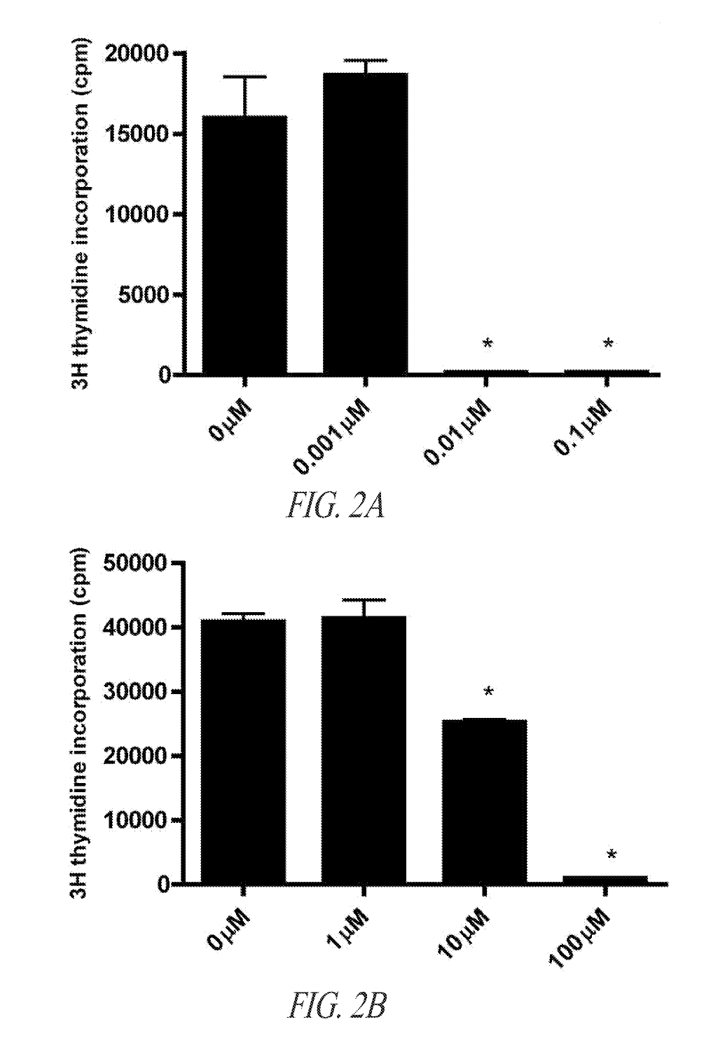

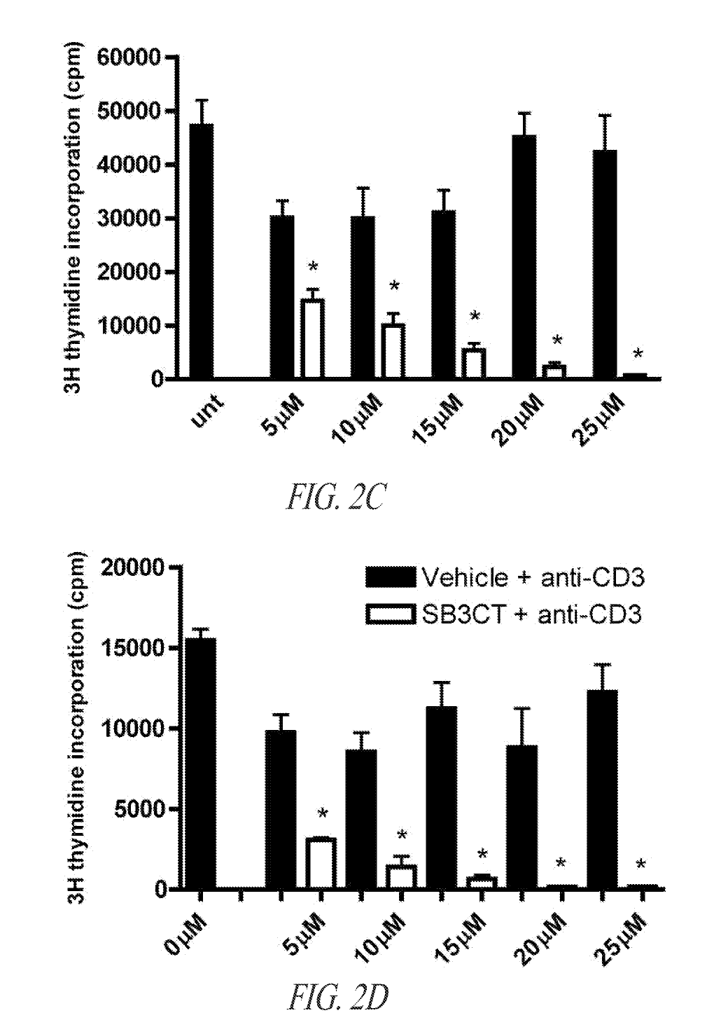

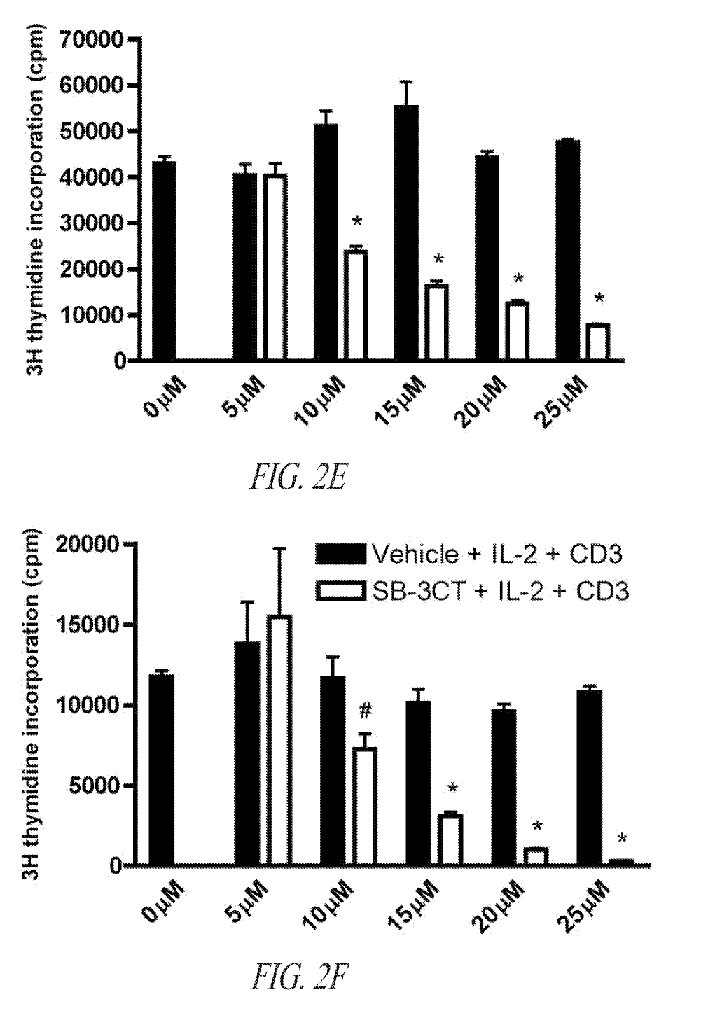

In order to determine the effects of MMPs on T cell activation, proliferation assays were utilized, in which, T cells were treated with 1,10-phenanthroline (a non-specific zinc chelator, 0.001-0.1 μM) and COL-3 (1-100 μM) for 6 hours, followed by stimulation with soluble anti-CD3 antibody for 72 hours. As shown in FIG. 2A, T cells treated with 0.001 μM of 1,10-phenanthroline displayed a proliferative response similar to untreated anti-CD3 antibody stimulated cells, whereas higher doses significantly abrogated the proliferative response (p<0.001). The suppressive effect observed at high phenanthroline concentrations was not due to toxicity as cells were viable after treatment. T cells treated with 1 μM of COL3 displayed a proliferative response similar to the untreated control. However, there was a dose-dependent decrease in T cell proliferation in response to higher doses (FIG. 2B) (p<0.001). Collectively, these data demonstrate that broad-spectrum MMP inhibition abrogates anti-CD3 antibody-induced T cell proliferation, suggesting an important role for MMPs in T cell activation.

### Example 3

### Highly Selective Inhibition of MMP2 and MMP9 Abrogate Anti CD3-Induced T Cell Proliferation

Previous studies that have utilized broad-spectrum MMP inhibitors (MMPI) have reported lack of specificity and negative effects on other non-MMP related signaling events (Sandler et al., 2005). Accordingly, the effects of COL-3 and 1,10-phenanthroline on T cell proliferation, described above, may have been due to non-MMP related activities. To circumvent these limitations, a highly selective MMP2 and MMP9 (gelatinase) inhibitor, SB-3CT, was utilized. This inhibitor is transformed in an enzyme-dependent process in the active sites of MMP2 and MMP9, (Brown et al., 2000; Toth et al., 2000) leading to tight-binding inhibition (Forbes et al., 2009). To investigate the effects of gelatinase-specific inhibition on T cell proliferation, CD4⁺ and CD8⁺ T cells were isolated from wild-type C57BL/6 mice and treated with SB-3CT, then cultured in the presence of soluble anti-CD3 antibody. Notably, SB-3CT treated CD4⁺ (FIG. 2C) and CD8⁺ (FIG. 2D) T cells exhibited a dose-dependent decrease in proliferation in response to anti-CD3 antibody stimulation, as compared to vehicle-treated cells. Additionally, to verify gelatinase inhibition at the protein level, MMP9 protein expression was measured by gelatin zymography. This experiment demonstrated that MMP9 expression was decreased in CD8⁺ T cells following treatment with SB-3CT (10 μM).

The data described in the Examples herein thus far raise the possibility that SB-3CT-induced cytotoxicity or anergy could account for the observed effects on proliferation. However, trypan blue exclusion along with annexin V staining, used to detect early cell death, revealed that cell viability was greater than 90 percent following treatment with SB-3CT, suggesting that the decrease in proliferative ability is not due to cell death. To assess whether SB-3CT treatment induced T cell anergy, T cell proliferation assays were utilized, in which exogenous IL-2 was added to vehicle or SB-3CT-treated T cells cultured in the presence of soluble anti-CD3 antibody. As shown in FIG. 2E-F, the addition of IL-2 induces partial recovery of the proliferative response in CD4⁺ and CD8⁺ T cells, however as the concentration of SB-3CT increases, proliferation continues to decrease in a dose-dependent manner. These data show that exogenous IL-2 partially recovered T cell proliferation, suggesting a possible role of anergy in gelatinase inhibitor-induced suppression of proliferation in T cells.

### Example 4

### Proliferation is Diminished in Gelatinase Deficient CD4⁺ and CD8⁺ T Cells

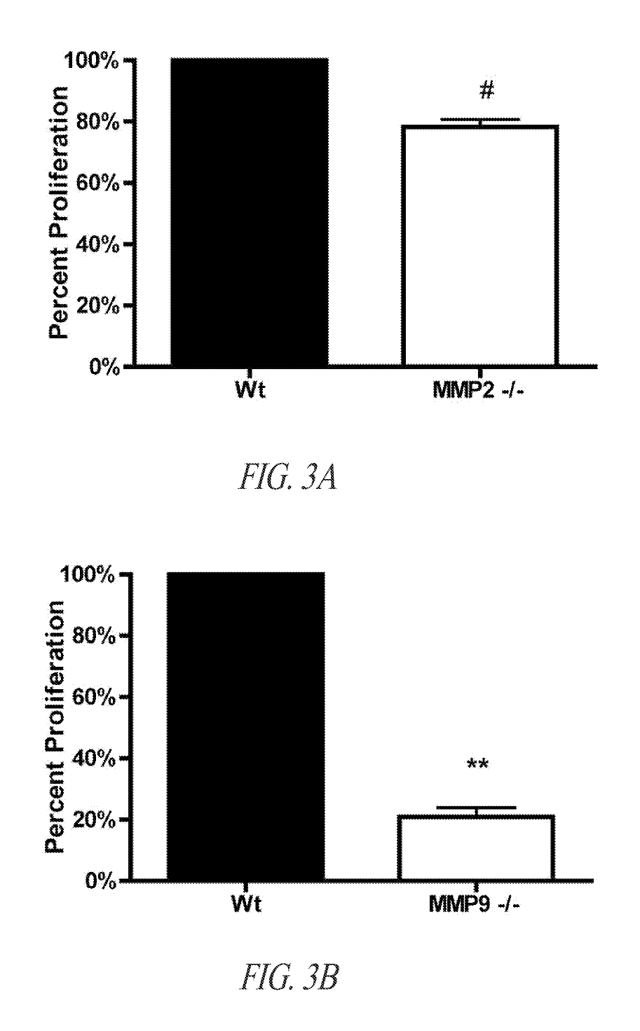

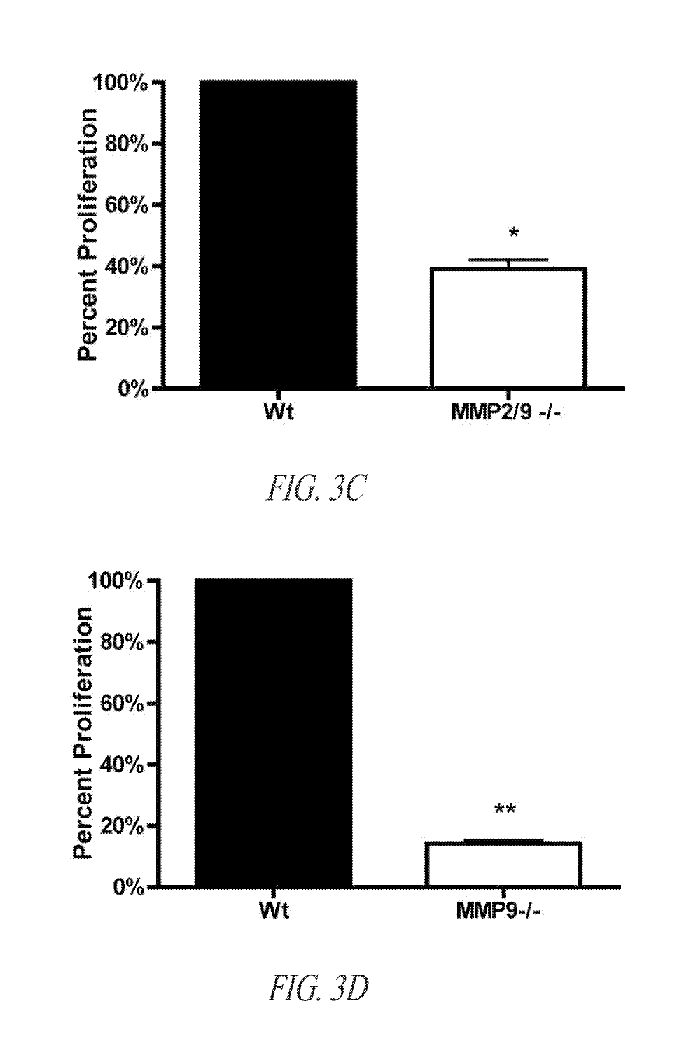

SB-3CT highly selectively inhibits MMP2 and MMP9 (Brown et al., 2000; Toth et al., 2000). Therefore, the effects of this inhibitor could have been due to blockade of either MMP2 or MMP9. To discern the roles of each MMP on T cell activation, anti-CD3 induced proliferation was examined in CD4⁺ T cells from MMP2−/−, MMP9−/−, or MMP2/9−/− mice. As compared to wild-type T cells, MMP2−/− CD4⁺ T cells only exhibited a 20% decrease in proliferation (FIG. 3A), whereas, MMP9 deficiency resulted in more than 80% reduction in proliferation (FIG. 3B) (p<0.001). Additionally, proliferation of MMP2/9−/− cells (double deficient) was intermediate to that of either MMP2−/− or MMP9−/− CD4⁺ T cells (FIG. 3C) (p=0.006). To determine if CD8⁺ T cells were also affected by MMP9 deficiency, the proliferative ability of MMP9−/− deficient CD8⁺ T cells was examined. The results revealed a >85% decrease in T cell proliferation (FIG. 3D) (p<0.001). These results confirm previous findings in SB-3CT-treated cells and indicate that MMP9, more so than MMP2, regulates proliferation of CD4⁺ and CD8⁺ T cells.

### Example 5

### Anti-CD3 Antibody-Induced Calcium Flux is Increased in MMP9 Deficient and SB-3CT-Treated T Cells

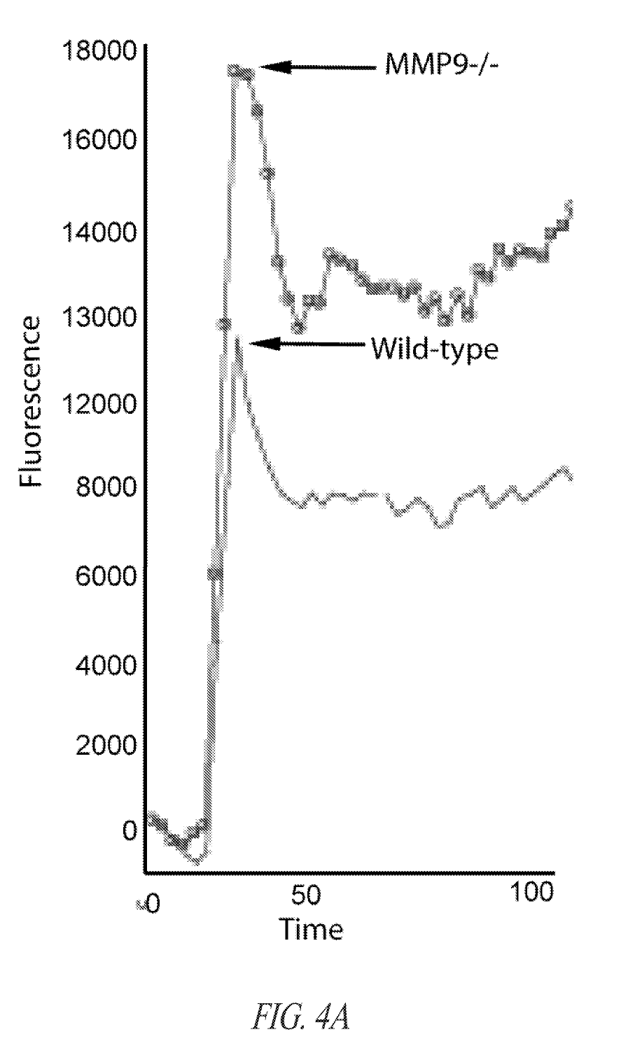

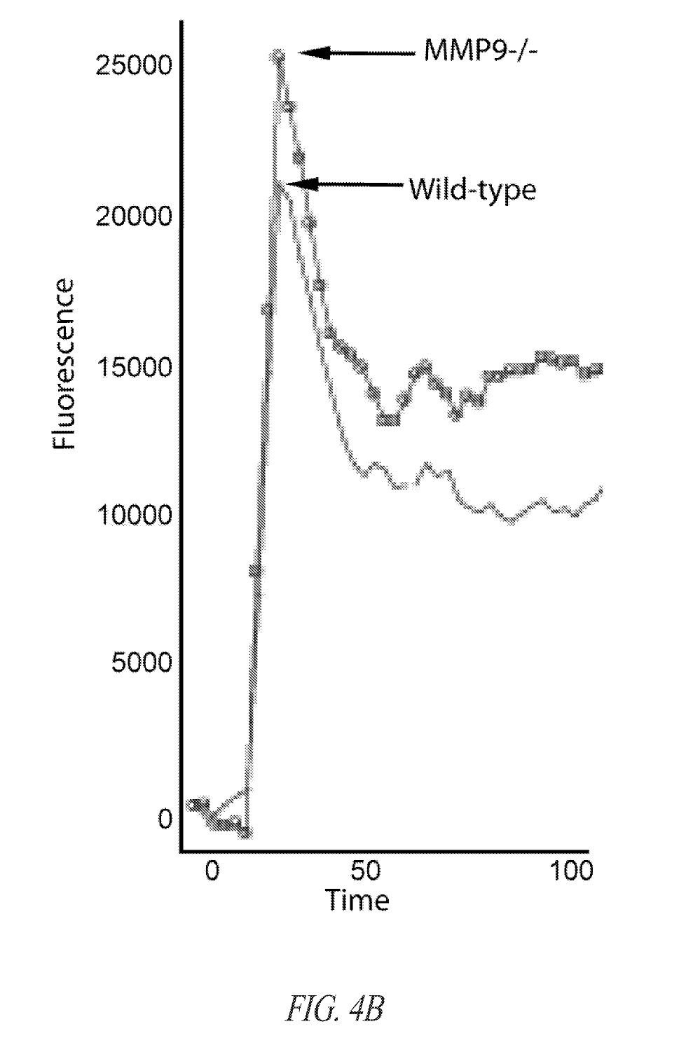

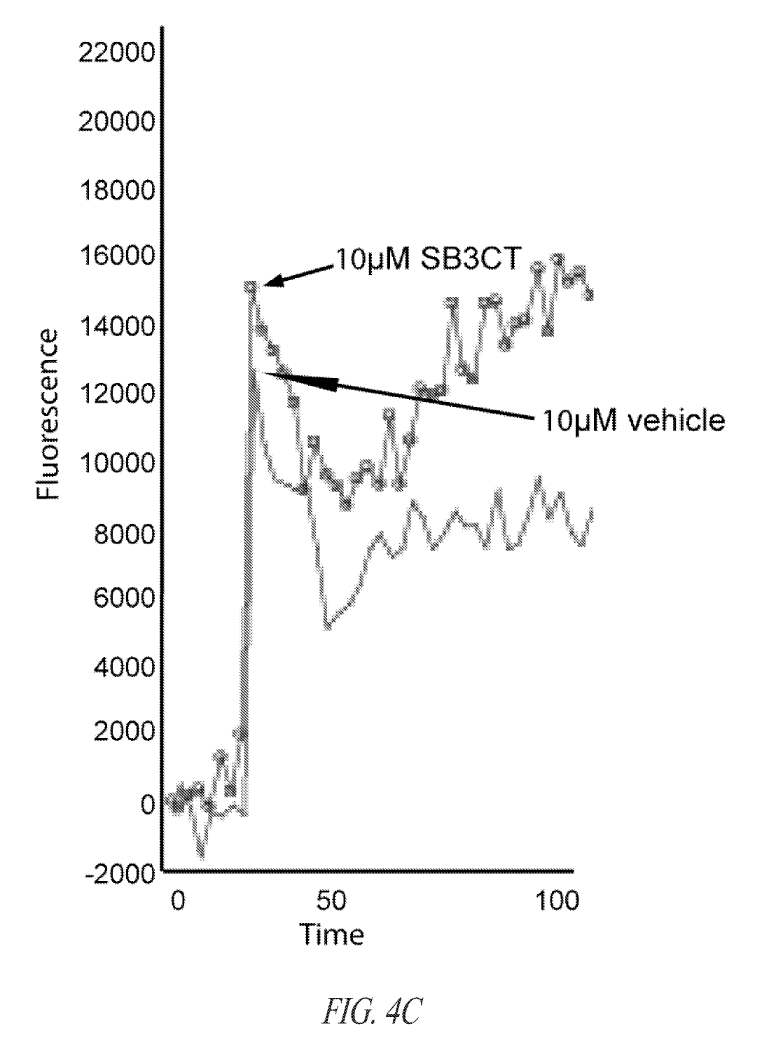

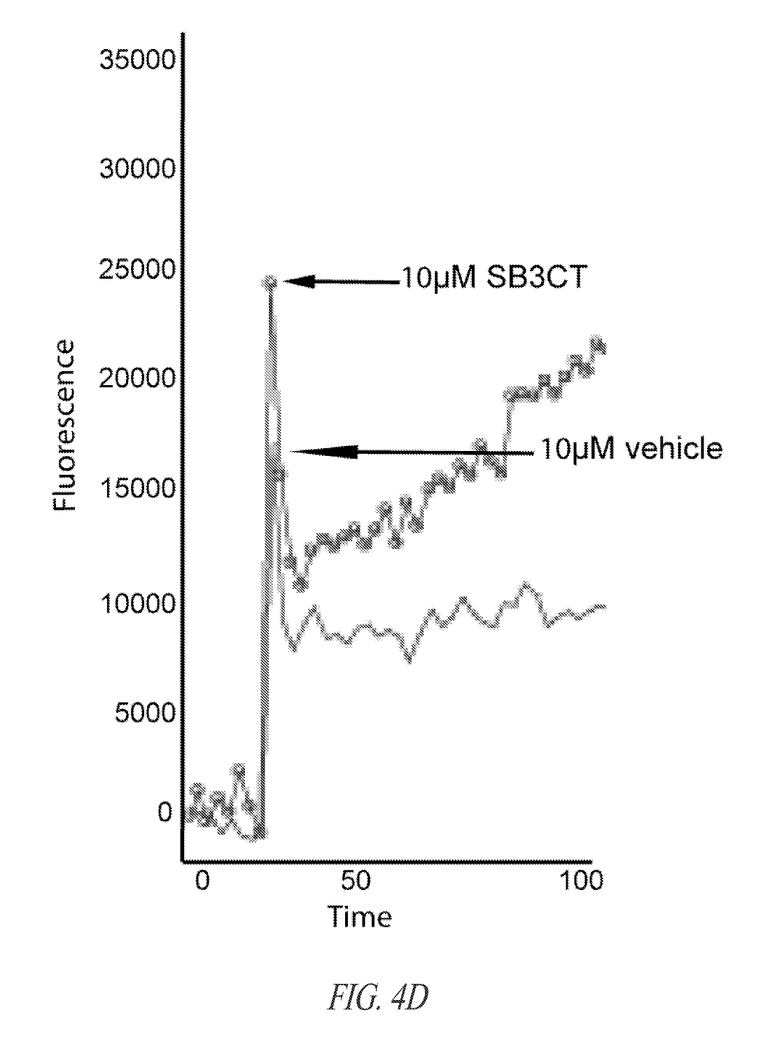

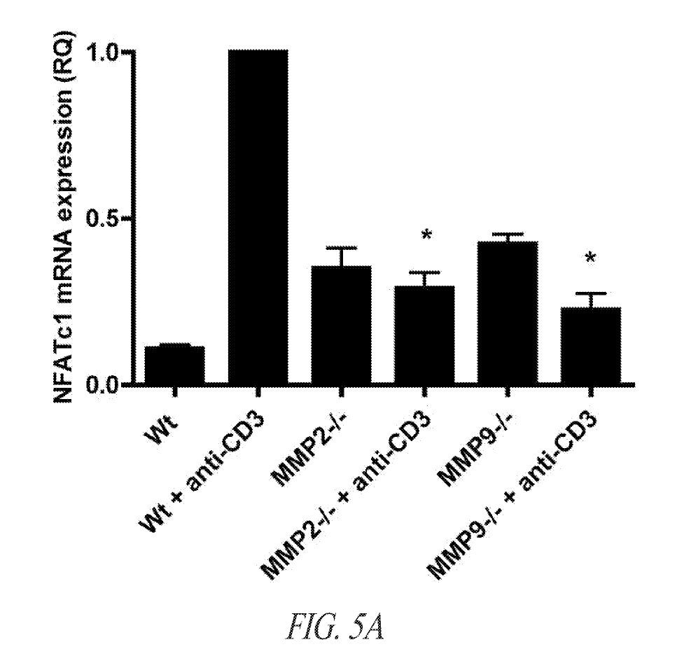

Since increased intracellular calcium flux is one of the early events post T cell receptor-mediated T cell activation (Hall and Rhodes, 2001; Zitt et al., 2004), the effect of MMP inhibition on intracellular calcium release from the endoplasmic reticulum (ER) was then examined. Since MMP9 deficiency had the greatest effect on T cell proliferation, anti-CD3-induced intracellular calcium flux was examined in MMP9−/− CD4⁺ and CD8⁺ T cells. Parallel studies were conducted examining wild-type CD4⁺ and CD8⁺ T cells treated with SB-3CT. Unexpectedly, MMP9−/− CD4⁺ and CD8⁺ T cells exhibited a greater degree of intracellular calcium flux, corresponding to release from the ER, as compared to wild-type control T cells (FIG. 4A-B). Similar to the results shown in MMP9−/− T cells, SB-3CT treatment also increased intracellular calcium flux corresponding to the release of calcium from the ER (FIG. 4C). To further examine the significance of gelatinase inhibition on anti-CD3 antibody induced calcium flux, it was determined if the presence of exogenous calcium in the media would alter the influx of calcium following SB-3CT treatment. Anti-CD3 treated wild-type CD8⁺ T cells were incubated in the presence of calcium containing media. As predicted, in the presence of calcium, not only was there an increase in calcium release from the ER (FIG. 4D), there was also a dramatic influx of exogenous calcium in SB-3CT-treated cells following anti-CD3 antibody stimulation. Taken together, these results demonstrate that MMP9 down regulates intracellular calcium flux in normal T cells in response to anti-CD3-induced activation.

### Example 6

### NFATc1 and CD25 mRNA Expression is Altered in MMP2- and MMP9-Deficient or SB-3CT-Treated T Cells

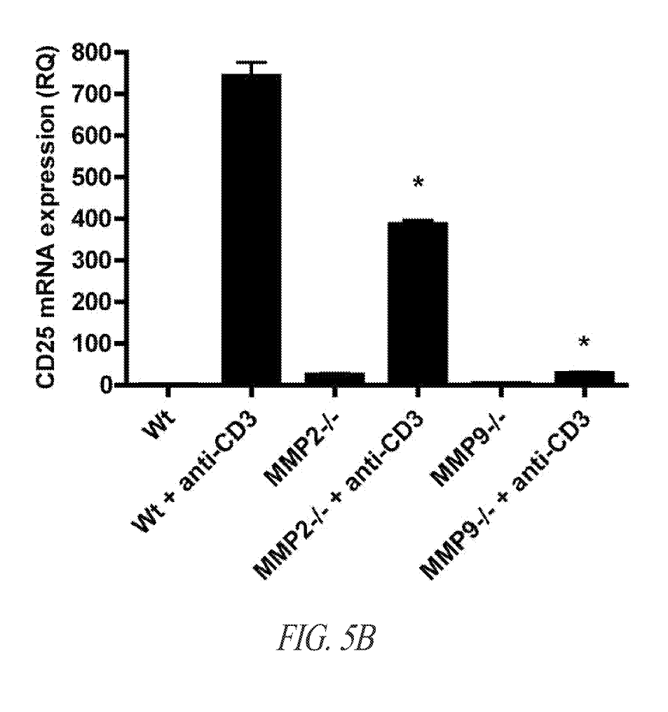

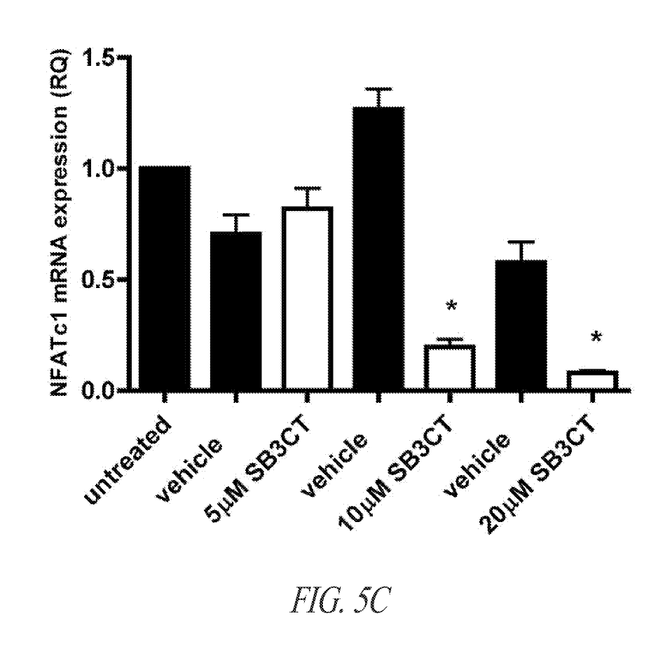

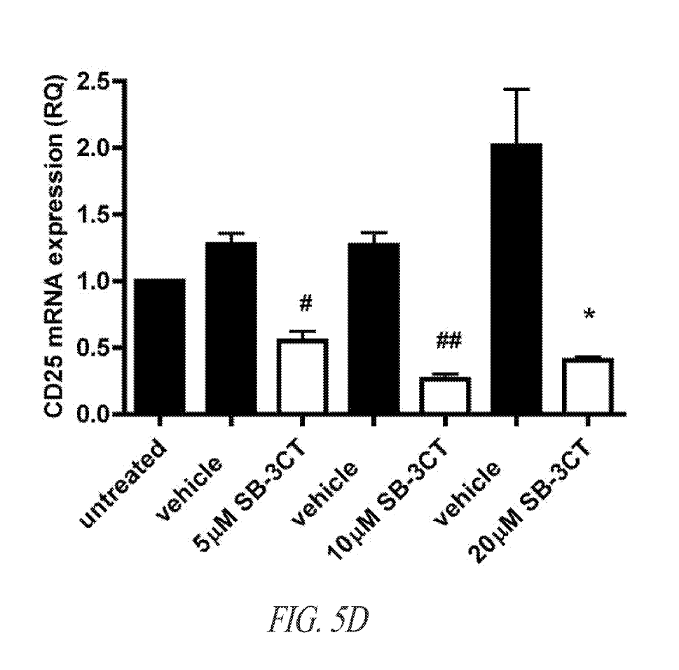

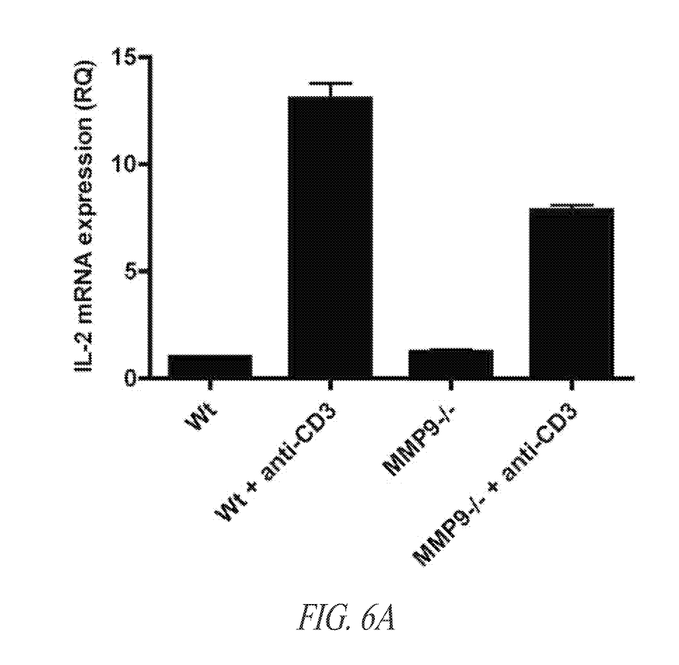

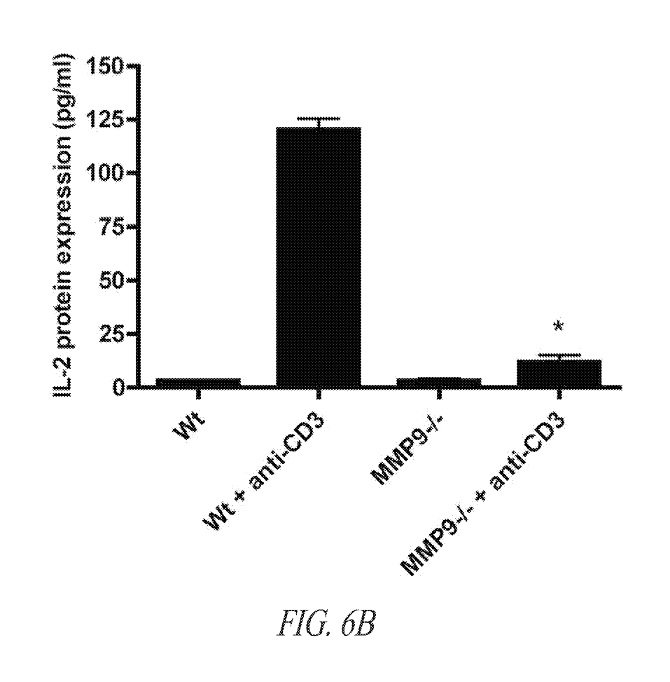

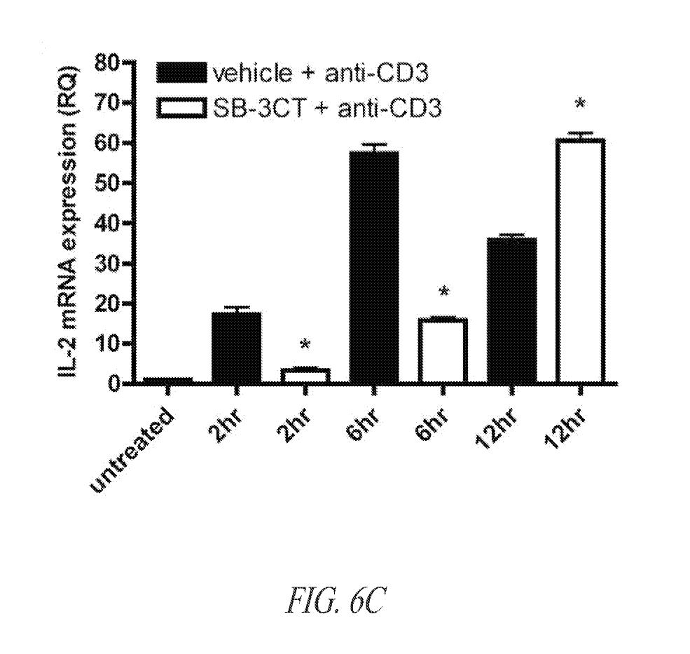

Following calcium signaling, nuclear factor of activated T cells (NFAT) nuclear translocation is critical for T cell activation and in promoting the transcription of IL-2Rα (CD25) and IL-2 expression (Yoshida et al., 1998). The effect of gelatinase deficiency and SB-3CT treatment on NFAT and CD25 mRNA expression in activated T cells was investigated. Since the data thus far show that CD4⁺ and CD8⁺ T cells respond similarly, CD4⁺ T cells were used in this next set of studies. MMP2−/− and MMP9−/− CD4⁺ T cells were stimulated with anti-CD3 antibody and NFATc1 and CD25 cytokine transcripts analyzed by quantitative RT PCR. Strikingly, MMP2−/− and MMP9−/− CD4⁺ T cells displayed a significant defect in their ability to express NFATc1 levels following anti-CD3 antibody stimulation, as compared to wild-type control T cells (FIG. 5A). Consistent with impaired induction of NFATc1, expression of CD25 transcripts, which is dependent on NFATc1, was also reduced significantly in both cell types and the reduction was greatest in MMP9−/− T cells (FIG. 5B).

These studies were also performed in CD4⁺ T cells following gelatinase inhibition by SB-3CT treatment. SB-3CT treatment abrogated NFATc1 and CD25 transcript expression in a dose-dependent manner, as compared to vehicle treated T cells (FIGS. 5C and 5D, respectively). Taken together, the decrease seen in NFAT and CD25 mRNA expression, both of which are regulated intracellularly, in response to gelatinase inhibition or absence suggests that gelatinases may regulate T cell activation by targeting an intracellular substrate, thereby preventing T cell activation.

### Example 7

### Cytokine Transcript and Protein Expression is Impaired in MMP9−/− and SB-3CT-Treated Wild-Type CD4⁺ or CD8⁺ T Cells

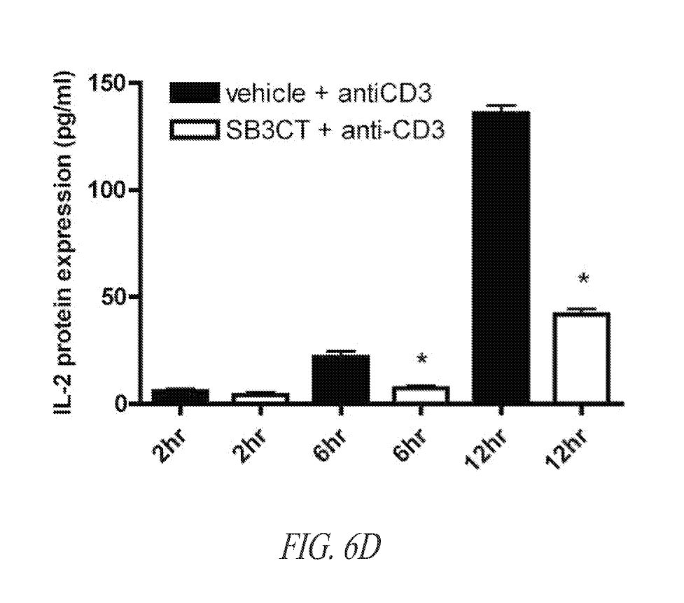

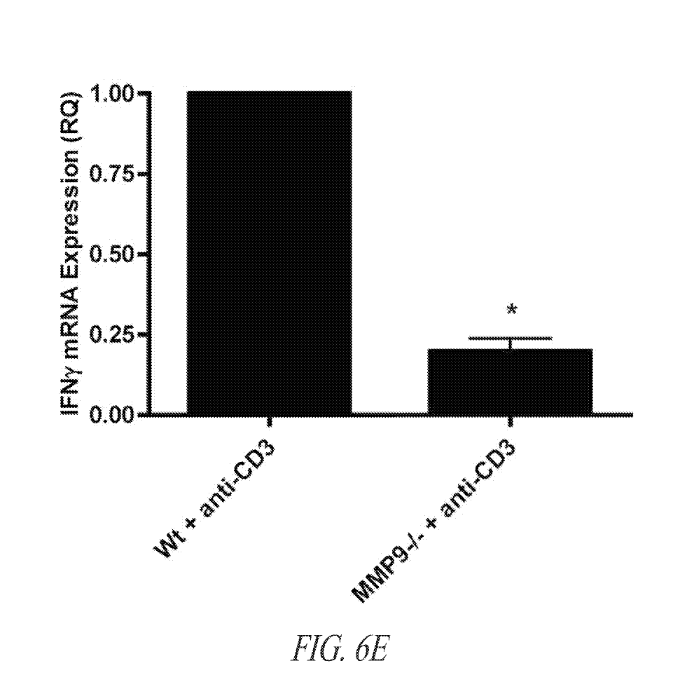

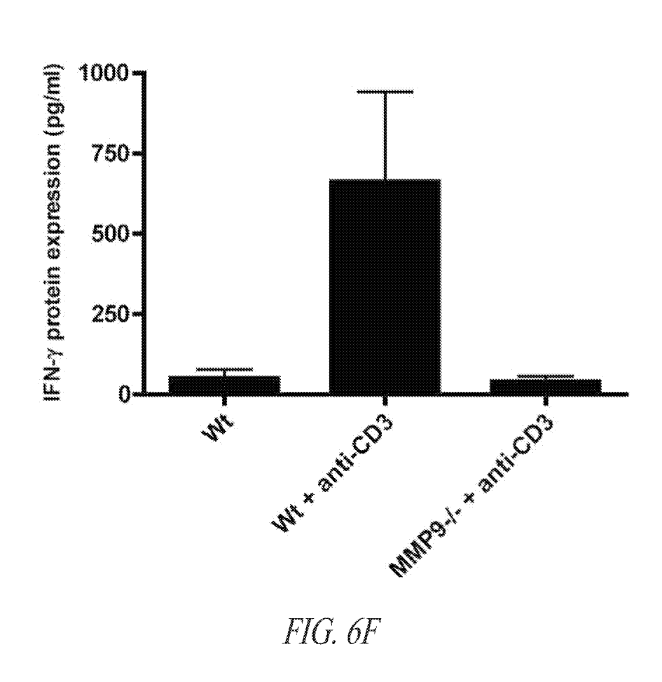

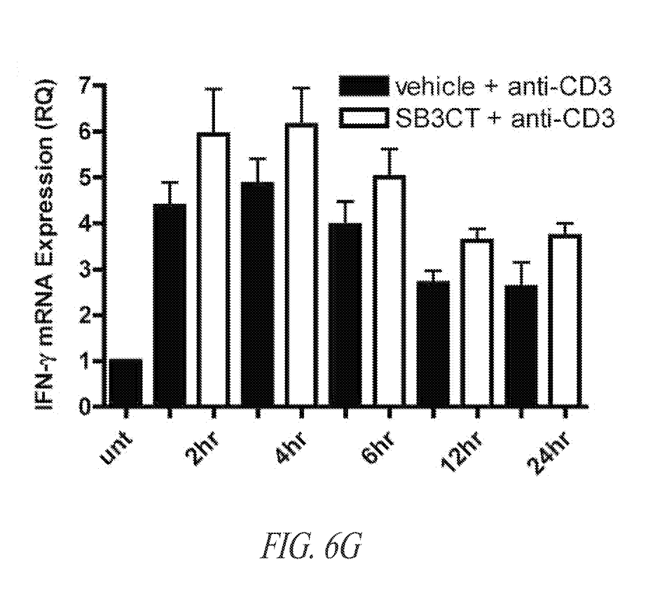

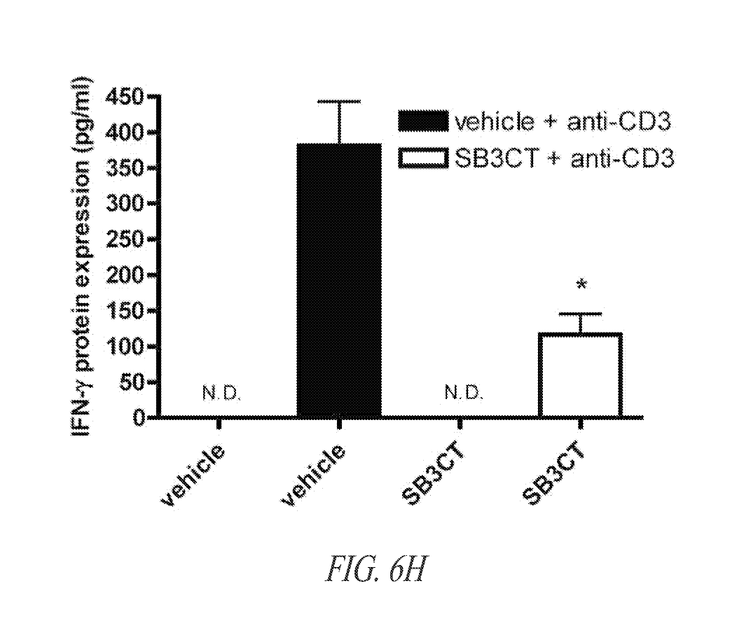

IL-2 and IFN-γ are produced in CD4⁺ and CD8⁺ T cells in response to anti-CD3 activation. The role of gelatinase inhibition or MMP9 deficiency was therefore determined in the expression of these cytokines. Notably, genetic deficiency in MMP9 significantly down regulated transcript and protein expression of IL-2 and IFN-γ in CD4⁺ (FIG. 6A-B) and CD8⁺ (FIG. 6E-F) T cells, respectively. The effect of gelatinase inhibition was examined at various time points on the expression of IL-2 and IFN-γ protein and transcript expression in the same cell types. Although IL-2 transcript expression increased over time in response to treatment with SB3CT, protein expression was down regulated (FIG. 6C, D). Similar trends were observed for IFN-γ in SB-3CT-treated cells (FIG. 6G, 6H).

### Example 8

### Gelatinase Inhibition Does Not Induce Regulatory T Cell Function

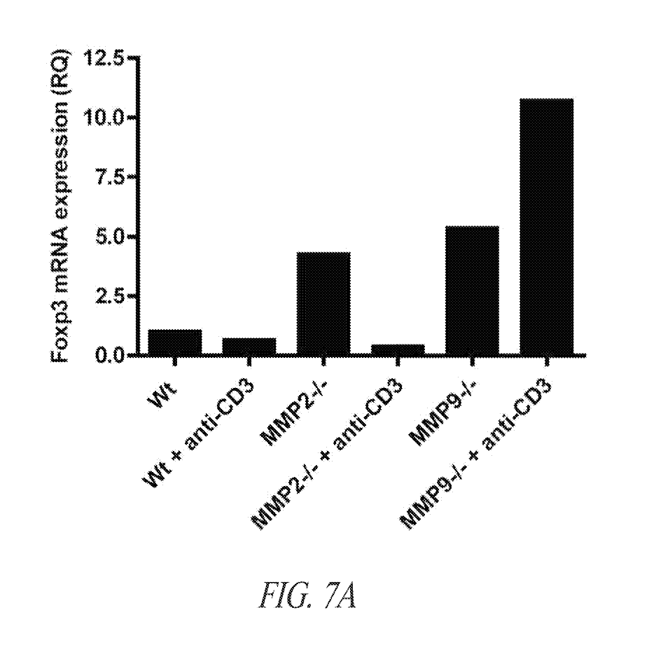

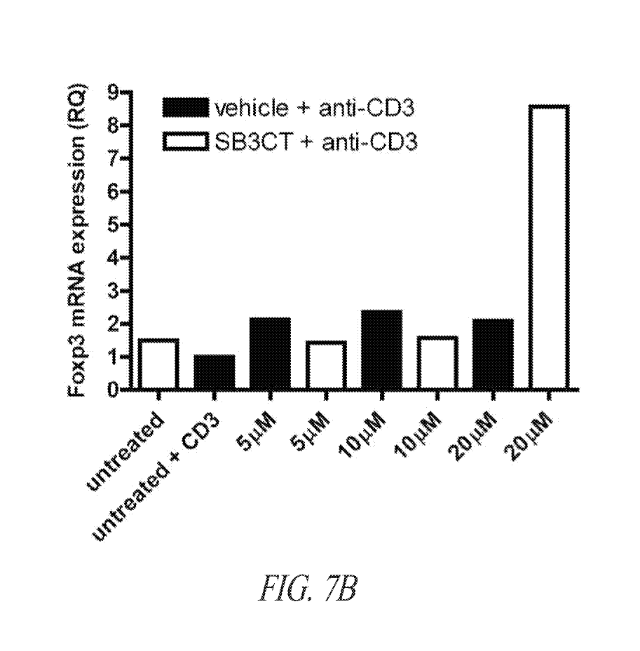

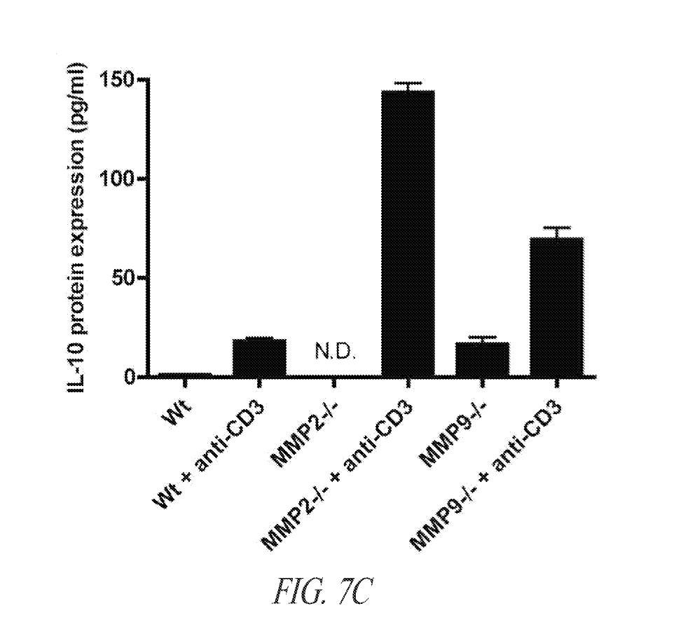

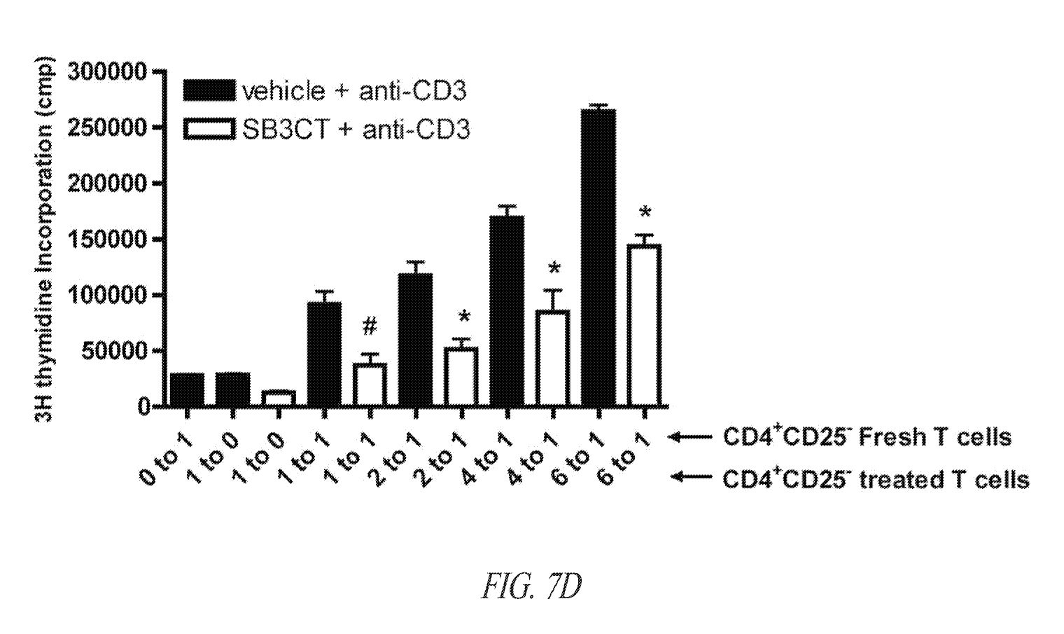

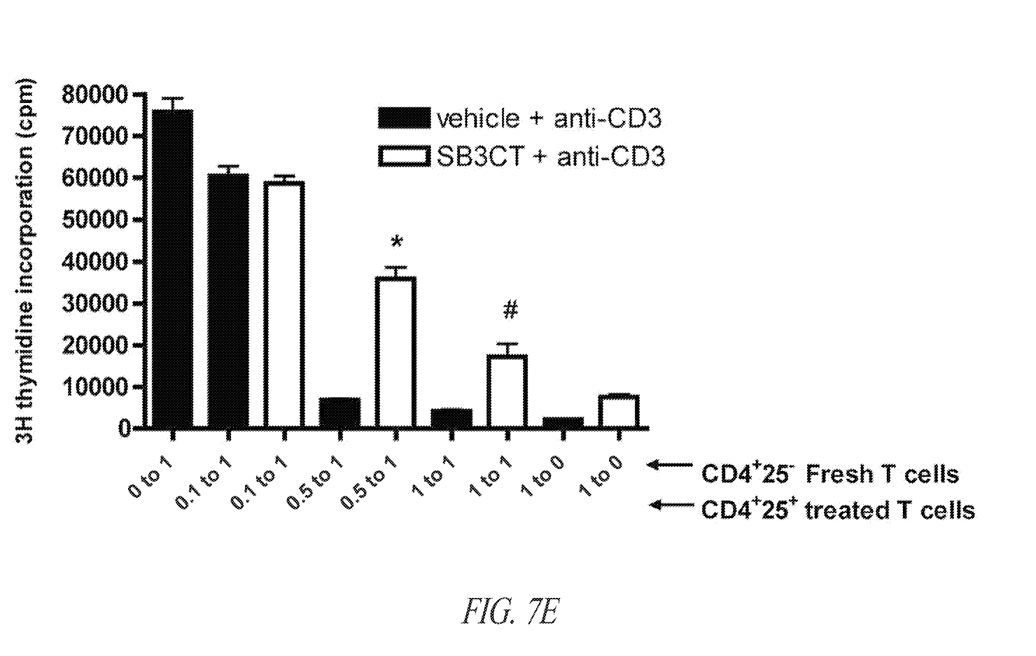

Studies have shown that regulatory T cells (Tregs) are unable to proliferate or produce IL-2 following anti-CD3 antibody stimulation, but are capable of suppressing proliferative responses and cytokine production by secreting IL-10 or up-regulation of forkhead transcription factor (foxp3), which inhibits NFAT expression (Thornton and Shevach, 1998). To determine if MMP9-deficient or SB-3CT-treated T cells exhibited Treg characteristics, foxp3 mRNA and IL-10 protein expression were examined in response to anti-CD3 stimulation. Foxp3 transcript levels were significantly increased in MMP9−/− CD4⁺ T cells, as compared to MMP2−/− and wild-type cells stimulated with anti-CD3 antibody (FIG. 7A). Additionally, foxp3 transcripts were also increased in response to SB-3CT (FIG. 7B). Similar to foxp3, IL-10 protein expression was increased in MMP2−/− and MMP9−/− CD4⁺ T cells (FIG. 7C). Collectively, these data suggest that gelatinase inhibition or deficiency may result in T cells with regulatory function.

To directly examine if gelatinase inhibition induced regulatory T cell function, suppressor assays were utilized in which CD4⁺25− T cells were treated with SB-3CT and co-cultured at varying ratios with untreated CD4⁺25− T cells in the presence of irradiated antigen presenting cells (APCs) for 72 hours. As shown in FIG. 7D, SB-3CT treatment at each ratio inhibited T cell proliferation by 50%. However, as the ratio of SB-3CT-treated cells increased, T cell proliferation also increased, suggesting that SB-3CT treatment does not induce regulatory T cell function.

To determine if Treg function was affected in response to SB-3CT treatment, CD4⁺25⁺ T cells (Tregs) were treated with SB-3CT and co-cultured at varying ratios as shown above in the suppressor assay. CD4⁺25⁺ T cells retained their suppressive function (FIG. 7E). Worth noting however is that SB-3CT-treated CD4⁺25⁺ T cells displayed a somewhat altered suppressive ability, requiring more treated cells to exhibit their suppressive nature. Taken together, these data suggest that MMP9 inhibition does not induce a mechanism of regulatory T cells despite an increasing expression of Foxp3 and IL-10. These data, however, suggest MMP9 involvement in Foxp3 and IL-10 expression.

### Example 9

### MMP9 Deficiency Alters CD4⁺ and CD8⁺ T Cell Phenotypes in Response to Anti-CD3

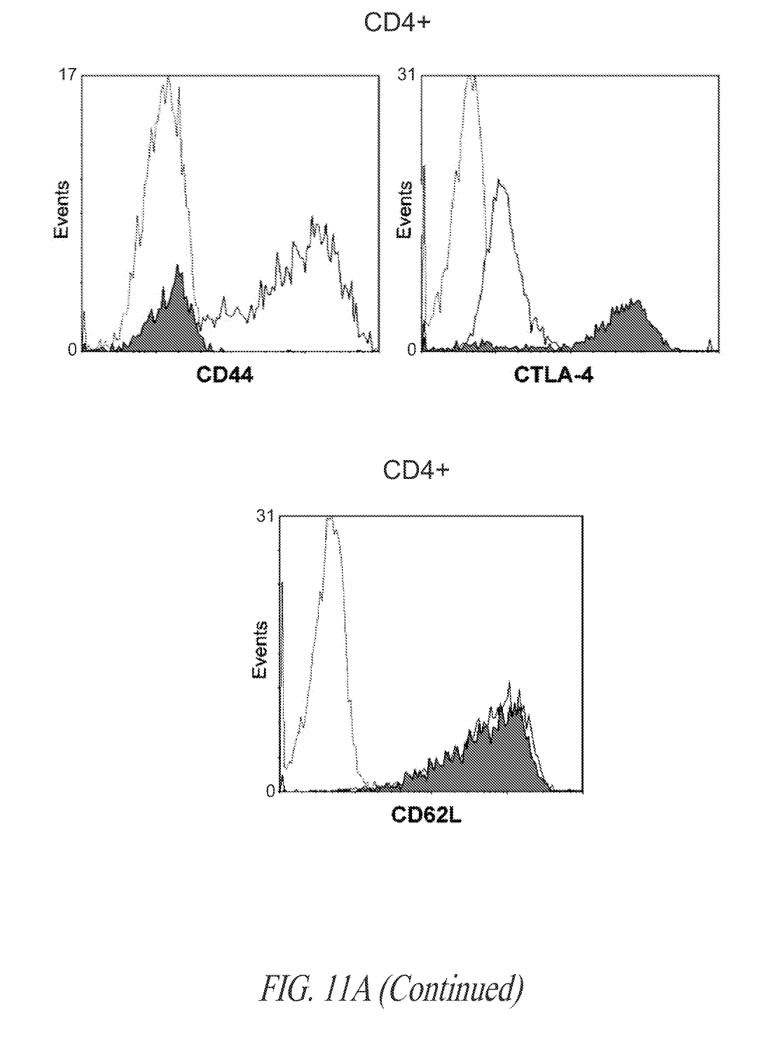

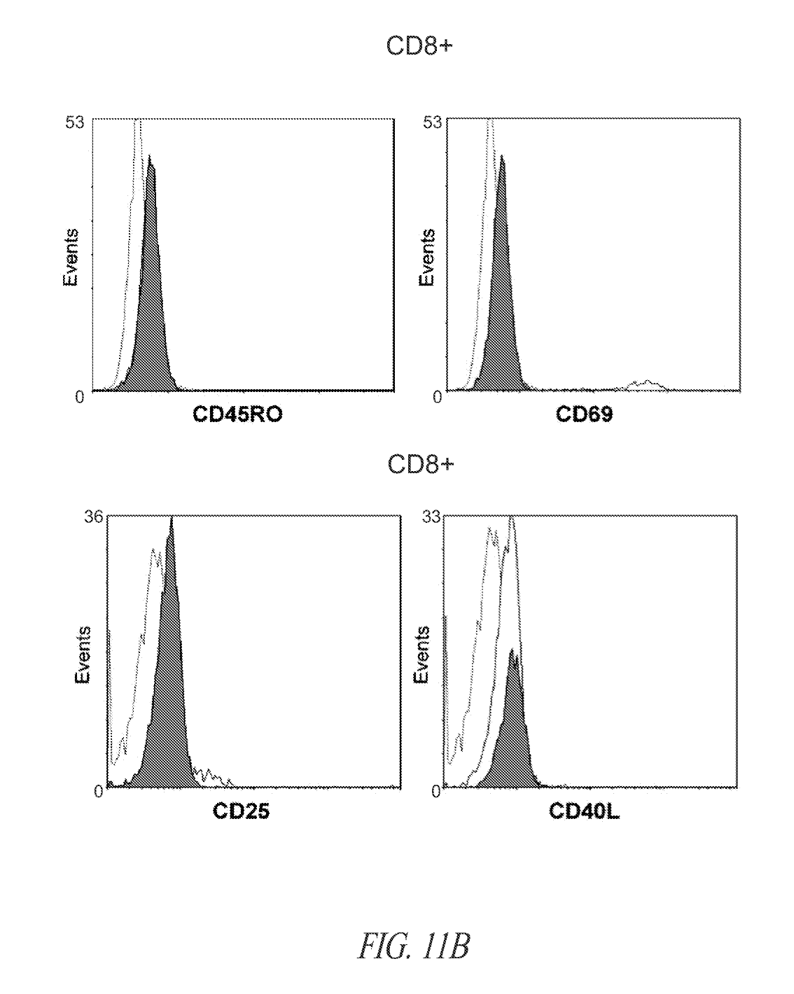

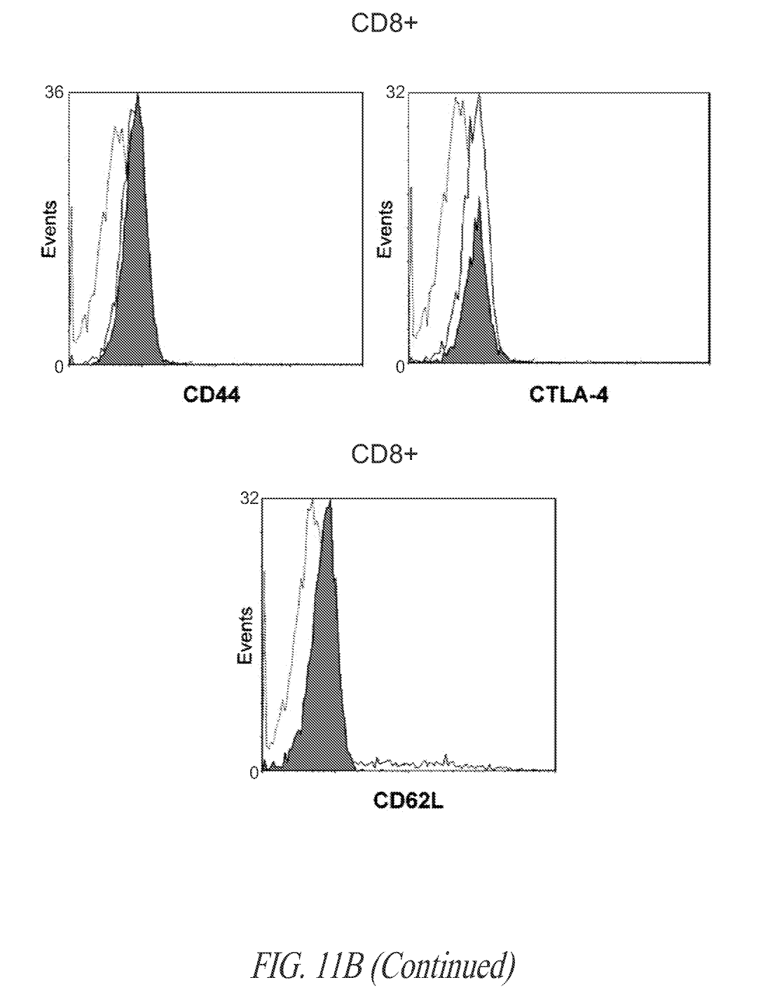

To further characterize the role of T cell derived MMP9, phenotype studies were performed on T cells in response to MMP9 absence (MMP9 deficient) by means of flow cytometry. A panel of seven T cell surface activation markers were assessed (Baroja et al., 2002; Bourguignon et al., 2001; Feng et al., 2002; Irie-Sasaki et al., 2003; Ivetic and Ridley, 2004; Leo et al., 1999; Stauber et al., 2006). CD4⁺ and CD8⁺ T cells isolated from wild-type and MMP9−/− C57BL/6 mice. MMP9 deficient or corresponding wild-type CD4⁺ or CD8⁺ T cells were cultured in the presence or absence of soluble anti-CD3 antibody and stained for various markers. Analysis of wild-type CD4⁺ T cells revealed increased surface expression levels of all of the T cell activation markers CD25, CD69, CD62L, CD44, CTLA-4, CD40L and CD45RO (FIG. 11 and Table 1). In comparison, analysis of CD4⁺ T cells from MMP9 deficient T cells revealed increased surface expression levels of CD62L, CTLA-4 and CD45RO. CD44 and CD40L expression levels decreased slightly, as compared to wild-type cells. CD25 and CD69 expression levels were both significantly diminished. These data show that as compared to wild-type CD4⁺ T cells, MMP9 deficient CD4⁺ T cells have significantly lower levels of cell surface CD25 and CD69, while expressing higher levels of CD45RO and CTLA-4.

TABLE 1
CD4⁺ and CD8⁺ MMP9−/− T cell activation marker expression

| | | MMP9−/− | | MMP9−/− |
|---|---|---|---|---|
| | Wt CD4+ | CD4+ | Wt CD8+ | CD8+ |
| | T cells | T cells | T cells | T cells |
| CD45RO | 17.90% | 98.20% | 5.00% | 5.40% |
| CD69 | 88.80% | 18.00% | 72.80% | 3.90% |
| CD25 | 92.80% | 31.60% | 63.80% | 11.90% |
| CD40L | 59.90% | 50.90% | 16.90% | 34.60% |
| CD44 | 97.60% | 77.50% | 20.10% | 24.00% |
| CTLA-4 | 62.30% | 96.20% | 14.90% | 25.10% |
| CD62L | 98.60% | 99.40% | 91.80% | 29.60% |

Anti-CD3 stimulated wild-type and MMP9−/− CD4⁺and CD8⁺ T cell surface expression of CD45RO, CD69, CD25, CD44, CD40L, CD62L, CTLA-4 was analyzed by flow cytometry. Data show the percent of positively stained cells shown in FIG. 11. Data are representative of two separate experiments.

Analysis of cell surface expression in wild-type CD8⁺ T cells revealed increases in CD25, CD62L and CD69 (FIG. 11 and Table 1). Additionally, CD40L, CD44 and CTLA-4 were expressed although the percent expression was less than or equal to 20%. CD45RO was also expressed at very low levels, not exceeding 5%. Analysis of MMP9 deficient CD8⁺ T cells as compared to wild-type CD8⁺ T cells revealed low expression levels of CD69, CD25, CD62L. CD45RO and CD44 surface expression levels remained the same as in wild-type cells. CTLA-4 and CD40L surface expression show slight elevation as compared to wild-type cells (FIG. 11 and Table 1). Consistent with the lack of induction of NFAT expression, CD25 expression did not increase in response to anti-CD3 stimulation in MMP9−/− T cells. Taken together, these data show that CD4⁺ and CD8⁺ T cells display differential cell surface expression in the absence of MMP9.

### Example 10

### Gelatinase Inhibition Abrogates Antigen-Specific CD8⁺ T Cell-Induced Lung Injury

The data have demonstrated that compared to CD4⁺ cells, CD8⁺ T cells express higher levels of MMP9 in response to anti-CD3, and that gelatinase inhibition or deficiency down regulates cellular function. Medoff et al. previously reported a murine model in which distal airway epithelial cells constitutively express OVA under the control of the CC10 promoter (CC10-OVA mice) (Medoff et al., 2005). Instilling activated CD8⁺ T cells that express an OVAspecific T cell receptor (OT-I) into the lungs of recipient mice, induces severe peribronchioloar inflammation (Medoff et al., 2005). Therefore, to examine the role of CD8⁺ T cell-derived gelatinases in vivo, the CC10-OVA murine model was utilized to determine if gelatinase inhibition in CD8⁺ T cells would down regulate lung injury (Stripp et al., 1992). To induce lung injury, CD8⁺ T cells were isolated from OT-1 transgenic mice, which have a TCR specific for the OVA peptide SIINFEKL bound to the class I MHC H-2 Kb and instilled into the lungs of CC10-OVA mice (Carbone and Bevan, 1989).

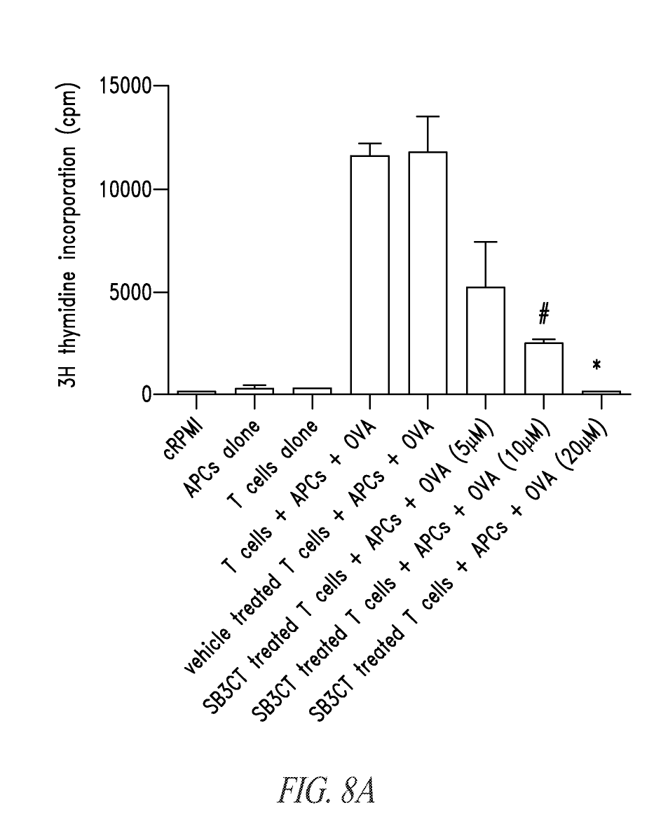

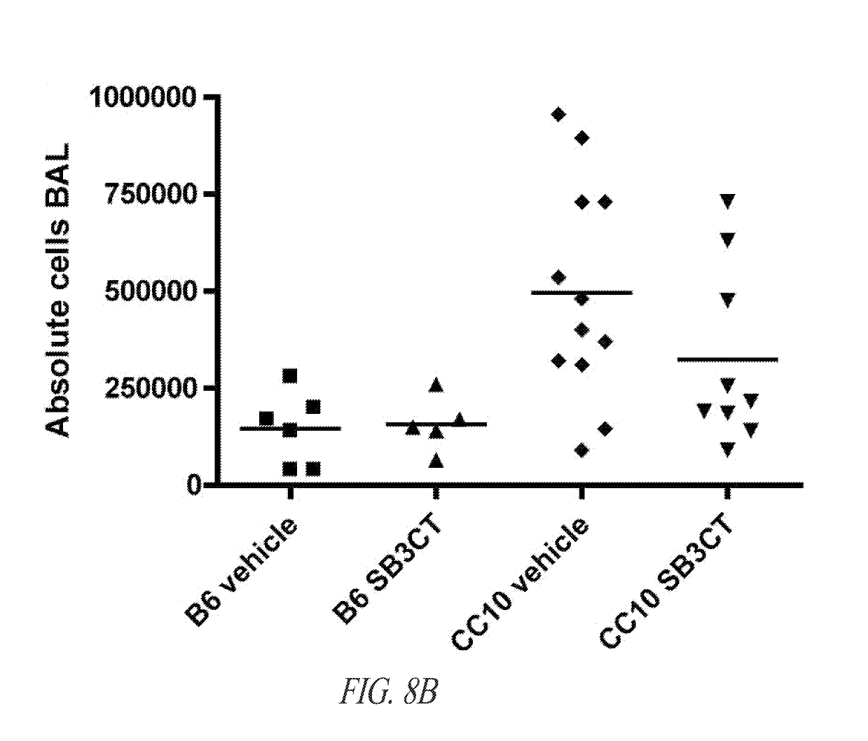

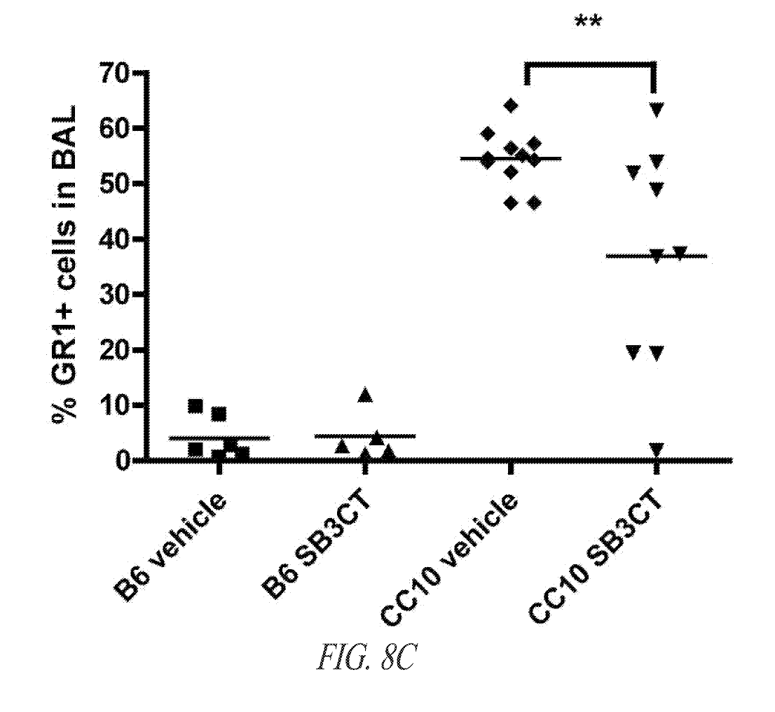

Studies in the prior Examples examined the effect of MMPs on polyclonal T cell activation via anti-CD3. To determined if highly selective gelatinase inhibition by SB-3CT would affect antigen-specific T cell proliferative function, OT-1 cells were treated with SB-3CT and cultured in the presence of peptide (SIINFEKL) pulsed antigen-presenting cells, as reported in methods. As shown in FIG. 8A, untreated or vehicle-treated OT-I transgenic CD8⁺ T cells proliferated in response to OVA peptide-pulsed antigen presenting cells. SB-3CT treatment of OT-I T cells completely abrogated the proliferative response to OVA pulsed antigen presenting cells. Examination of CD4⁺ T cells from OT-II transgenic mice revealed a similar trend. These data demonstrate that similar to polyclonal activation via anti-CD3, highly selective gelatinase inhibition also abrogates antigen-specific proliferation of CD8⁺ T cells.

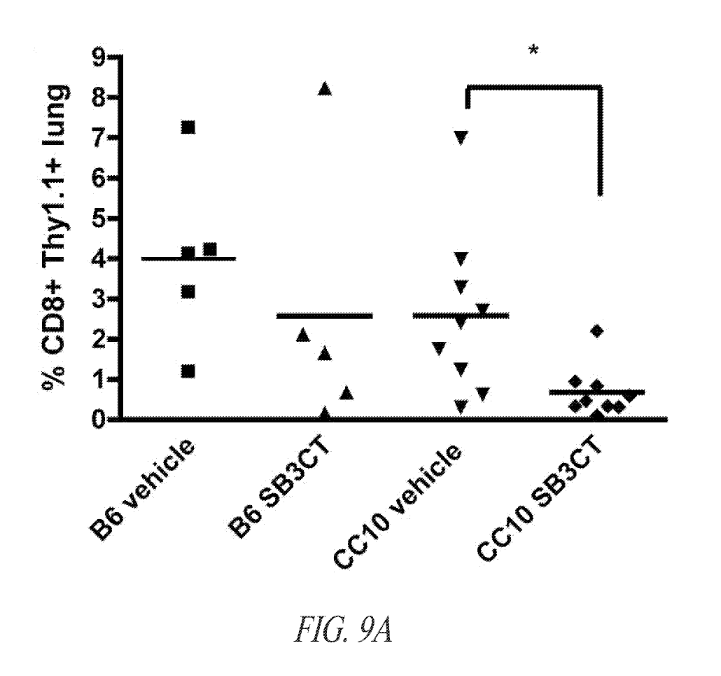

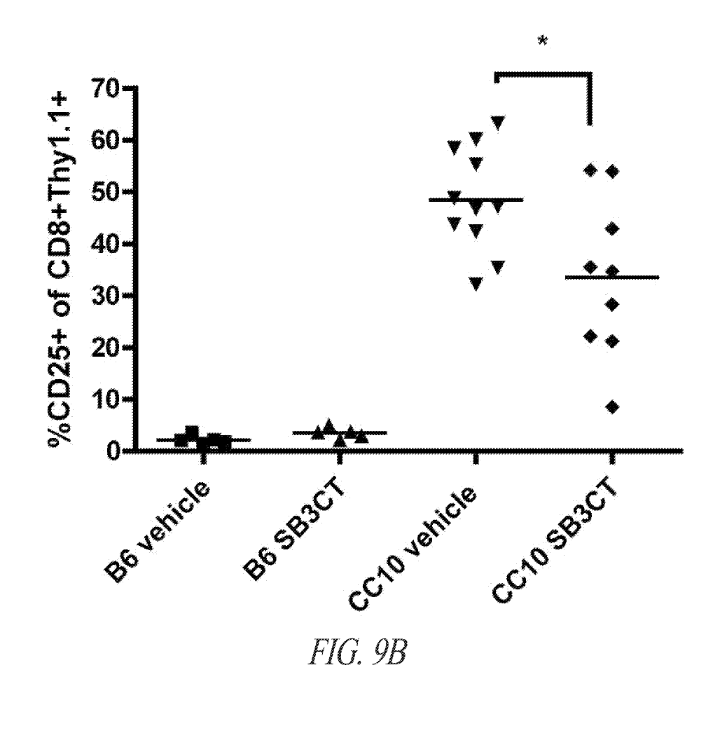

To determine whether gelatinase inhibition had an effect on antigen-specific T cell mediated lung injury in vivo, anti-CD3 and SB-3CT-treated OT-I CD8⁺ T cells were activated in vitro in the presence of OVA as described elsewhere herein and prior studies (Medoff et al., 2005). The cultured OT-I CD8⁺ T cells were transferred intratracheally into the lungs of CC10-OVA transgenic or non-transgenic wild-type C57BL/6 mice. Analysis of total cell accumulation in bronchoalveolar lavage seven days after adoptive transfer revealed no differences in the quantity of total BAL cells recovered in the SB-3CT-treated (MMPI) and vehicle groups (FIG. 8B). However, the quantity of neutrophils (Gr-1⁺), a marker of injury in this model (Medoff et al., 2005), was decreased significantly in the SB-3CT-treated group (FIG. 8C) (p<0.01). The OT-I transgenic mice were Thy1.1⁺ and therefore, provided a means of tracking the transferred cells in the CC10-OVA mice, which were in a Thy1.2⁺ background. Next, it was determined if there was a difference in the accumulation of CD8⁺ Thy1.1⁺ T cells in the lung between the two CC10-OVA treated groups (vehicle or SB-3CT). Treatment with SB-3CT resulted in significantly fewer CD8⁺ Thy1.1⁺ (donor) cells in lung parenchyma (FIG. 9A) (p<0.01). Moreover, fewer of these cells expressed the activation marker CD25 (FIG. 9B) (p<0.01).

Fewer neutrophils and donor derived CD8⁺ T cells in lungs of CC10-OVA mice that received gelatinase-inhibited cells suggests less severe lung injury. Indeed, gelatinase inhibition of OT-I T cells prior to adoptive transfer abrogated the development of perivascular and peribronchiolar inflammation as shown by histology of the lungs evaluated by H&E staining.

### Discussion

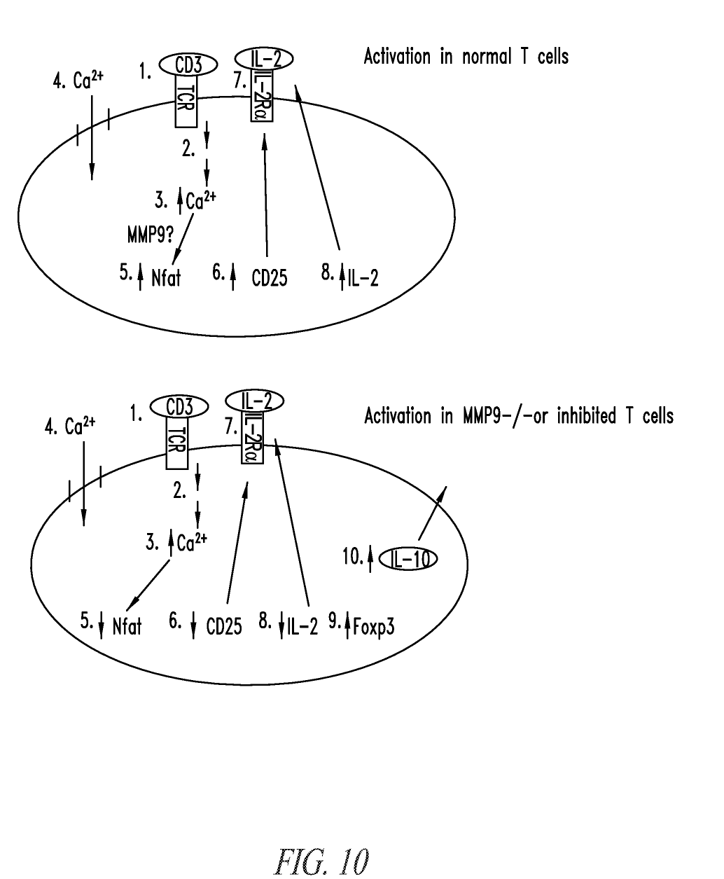

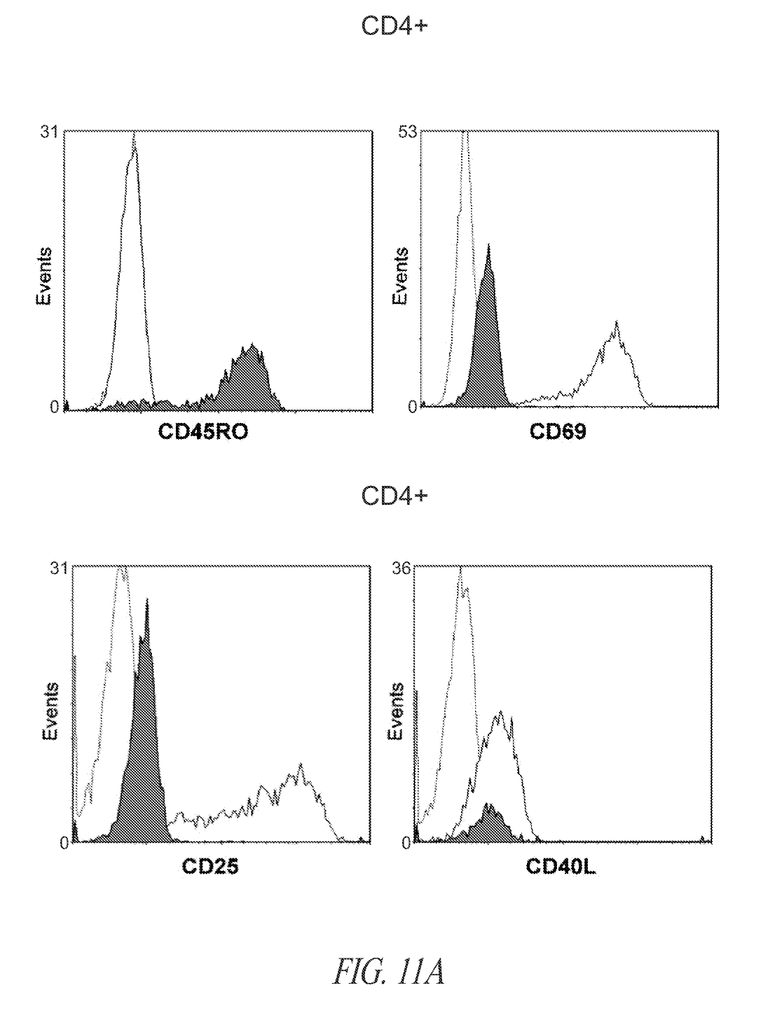

Data from the current study reveals that MMP9, in particular, plays a key role in regulating T cell activation. This conclusion is derived from data showing that MMP9 inhibition significantly impairs the activation of CD4⁺ and CD8⁺ T cells. However, it is notable that MMP9 is induced greatly in activated CD8⁺ compared to CD4⁺ T cells. In the current study it is shown that broad-spectrum MMP inhibition, MMP9-specific inhibition, as well as genetic deficiency of MMP9, all result in down regulation of polyclonal activation-induced proliferation in CD4⁺ and CD8⁺ T cells. NFATc1 and CD25 gene expression were down-regulated, while foxp3 gene expression and IL-10 protein expression levels were elevated. Analysis of IL-2 and IFN-γ cytokine gene and protein expression revealed down-regulation of gene and protein expression in response to MMP9 inhibition and MMP9 deficiency. However, gelatinase deficiency or inhibition was associated with increases in intracellular calcium release in response to polyclonal stimulation via anti-CD3 (FIG. 10). It was also demonstrated in an in vivo model that MMP9 inhibition impaired the degree of T cell mediated lung injury. Collectively, these data clearly indicate a role for T cell derived MMP9 in the process of T cell activation.

Recently, reports have begun to show a functional role of MMPs in allograft rejection and their role in T cell alloreactivity. Fernandez et al. reported in a tracheal allograft obstructive airway disease (OAD) model, that MMP9-deficient host mice did not develop OAD but exhibited enhanced T alloreactivity (Fernandez et al., 2005). In the present studies however, it is disclosed that MMP9 deficiency significantly abrogated T cell proliferation. One reason for these dissimilar results may be due to the fact that in the OAD model, bulk T cells (CD3⁺) were stimulated with allogeneic DCs, thereby inducing non-specific T cell activation. In the present studies, however, MMP9-deficient CD4⁺ and CD8⁺ T cells were cultured separately in the presence of anti-CD3 antibody, allowing individual examination of how these two cell populations function in the process of T cell activation. It has been reported that T cells and macrophages are important to the development of OAD (Kelly et al., 1998; Neuringer et al., 2000), as studies have shown that mice with a genetic T cell deficiency, such as severe combined immunodeficient (SCID) mice or recombinase activating gene 1-deficient (RAG−/−) do not develop OAD (Neuringer et al., 1998). These studies provide strong evidence that T cells are important in the development of OAD and suggest that T cell derived MMP9 may play an important role in this development. Thus, inhibiting T cell derived MMPs can result in decreased T cell activation, which may provide protective effects in response to a variety of pathogenic states.

In the investigation of the intracellular T cell signaling events, it is disclosed herein that in response to gelatinase absence or inhibition, T cells displayed increased levels of calcium release from the ER as well as exogenous calcium influx following anti-CD3 antibody stimulation. These findings suggested that in response to MMP9 inhibition or MMP9 deficiency the increase in calcium influx may be a mechanism by which a cell attempts to compensate for the lack of effective activation events. Accordingly, MMP9 may function as a tonic down-regulator of calcium mediated events. Further downstream, the results showed that NFAT gene expression was abrogated in MMP9-deficient or SB-3CT-treated T cells.

Due to the importance of NFAT as a transcription factor in T cell activation, it is likely that alteration of NFAT expression alters the expression of other NFAT-dependent genes such as IL-2Rα (CD25) and IL-2 that rely on NFAT translocation for their proper function. Indeed, a decrease in CD25 mRNA and surface expression in MMP9-deficient and SB-3CT treated T cells was observed. These findings strongly suggest that gelatinase inhibition down-regulates NFAT activation, possibly by repressing NFAT transcription, which in turn decreases CD25 and IL-2 expression. The decrease in CD25 expression means that less CD25 will be present on the cell surface, which will limit the number of receptors available to bind IL-2 and induce proliferation, thereby abrogating T cell activation. This may explain why the addition of exogenous IL-2 did not recover the proliferative response in SB-3CT-treated cells as shown in FIG. 2. Since the results suggested that gelatinase inhibition may cause the T cells to exhibit Treg function, targets that are characteristically found in Tregs were investigated. Unexpectedly, it was observed that foxp3 expression was elevated in SB-3CT-treated and MMP9-deficient T cells. In T cells that have adopted the Treg lineage, the inability to produce IL-2 and IFN-γ, seems to be a consequence of transcriptional repression by foxp3 (Chen et al., 2006; Lee et al., 2008; Marson et al., 2007; Wu et al., 2006). The present studies demonstrated decreased levels of IL-2 and IFN-γ. Therefore, foxp3 may be actively repressing IL-2 and IFN-γ gene expression in response to TCR ligation, thereby causing a decrease in T cell activation. Since IL-10 is a characteristic immunosuppressive cytokine secreted by Tregs and Tr1 cells, IL-10 protein expression was assessed in MMP9-deficient T cells and reported that IL-10 was elevated in MMP9-deficient T cells following stimulation with anti-CD3 antibody. Gelatinase inhibition did not induce regulatory T cell function. These results may suggest that inhibition of MMP9 leads to the development of a new IL-10 secreting T cell subset that exhibits regulatory T cell characteristics, but not regulatory T cell function. Although MMP9 inhibition did not induce regulatory T cell function, Treg function was altered in response to MMP9 inhibition. A report by Pan et al. demonstrated that Eos, a zinc-finger transcription factor mediates foxp3-dependent gene silencing in Tregs (Pan et al., 2009). In the present disclosure, MMP9 inhibition may induce Eos, which may mediate foxp3-dependent suppression of IL-2 and IFN-γ, thereby causing the decrease in normal T cell activation.

In the investigation of gelatinase inhibition in vivo, a significant decrease in the percentage of CD8⁺ Thy1.1⁺ T cells in the lung of CC10-OVA mice was observed, suggesting that gelatinase inhibition may affect T cell migration and/or decrease cellular activation. Further analysis of CD25 surface expression on CD8⁺ Thy1.1⁺ T cells in the lung revealed a dramatic decrease in CD25 surface expression suggesting decreased cellular activation. These results are similar to the in vitro data demonstrating a significant decrease in CD25 mRNA and cell surface expression in response to gelatinase inhibition. Histological analysis of lung sections collected from the lungs of CC10-OVA mice demonstrated increased perivascular and perinuclear infiltrates following the transfer of vehicle-treated OT-1 cells. In contrast, following the adoptive transfer of SB-3CT-treated OT-1 cells, the mononuclear cellular infiltration was minimal, suggesting that MMP9 inhibition attenuated the degree of inflammation within the lung, thus significantly impairing the degree of T cell-mediated lung injury.

The present results strongly indicate that MMP9 plays a definite role in T cell activation and are suggestive that this role is intracellular by modulation of mRNA and protein expression.

The present studies reveal a critical role for functional T cell-derived gelatinases in activating CD4⁺ and CD8⁺ T cells and suggest that gelatinase inhibition could be a novel approach to immunosuppression for the treatment of T cell-dependent diseases such as organ allograft rejection and autoimmune diseases.

The experiments described in the Examples herein were carried out using the following methods.

Animals

Female Balb/c and C57BL/6 mice 6-10 weeks old, were purchased from Harlan (Indianapolis, Ind.) or bred independently. MMP2 deficient (MMP2−/−), MMP9 deficient (MMP9−/−) and MMP2/MMP9 double deficient (MMP2/9−/−) mice (C57BL/6 background) (Baylor College of Medicine, Houston, Tex.), CC10-OVA mice (C57BL/6 background) and OT-1 TCR transgenic mice (C57BL/6-Thy1.1 background) were also utilized (Corry et al., 2004; Shilling et al.). All mouse studies were conducted in accordance with institutional animal care and usage guidelines.

T Cell Isolation

Single cell suspensions were prepared from the spleens of five to seven mice. Red blood cells were lysed with an NH4Cl lysis buffer. CD4⁺ and CD8⁺ T cells were then isolated using mouse CD4 (L3T4) and CD8 (CD8a-Ly2) Microbeads (Miltenyi Biotech, Auburn Calif.) per manufacturer's instructions. The purity of CD4⁺ and CD8⁺ T cells, determined by flow cytometry, ranged from 97 to 99%. This isolation protocol was used to isolate T cells from C57BL/6 wild-type mice, MMP2 deficient, MMP9 deficient, MMP2/9 deficient, OT-I transgenic and OT-II transgenic mice. Regulatory T cells (Tregs) were isolated using mouse CD4⁺ CD25⁺ Isolation Kit (Miltenyi Biotech, Auburn, Calif.). Treg cell purity determined by flow cytometry, exceeded 93%. Where indicated, the CD4-cell fraction was γ-irradiated (2000 rads) and used as antigen presenting cells.

Preparation of Matrix Metalloproteinase Inhibitors (MMPIs)

The non-specific MMP inhibitor, 1,10-phenanthroline (Sigma, St. Louis, Mo.) was reconstituted to 1 M solution in dimethyl sulfoxide (DMSO) and diluted to 0.001-0.1 μM in complete RPMI (cRPMI), composed of RPMI, 400 mM L-glutamine, 100 U penicillin streptomycin (Gibco, Carlsbad, Calif.), 10% FCS (Hyclone, Logan, Utah), and 5×10⁻⁵ M 2-mercaptoethanol (Sigma, St. Louis, Mo.). COL-3 is a chemically modified tetracycline and non-specific MMP inhibitor (CollaGenex Pharmaceuticals, Inc., Newtown, Pa.). COL-3 was reconstituted in DMSO to a 1 M solution then diluted to 1-100 μM in cRPMI. SB-3CT is a specific mechanism-based MMP2/9 inhibitor and was reconstituted in DMSO and polyethylene glycol (PEG) to a 1 M solution then diluted to 0.0001-1 mM in cRPMI.

T Cell Proliferation Assays

CD4⁺ or CD8⁺ T cells were isolated from wild-type Balb/c or C57BL/6 mice (1×10⁶/ml) and incubated with the indicated concentrations of MMPIs or vehicle control for 6 hours. The treated cells were then washed three times in RPMI and cultured (1×10⁵/well) in a 96 well plate in 200 μl of cRPMI in the presence of anti-CD3 antibody (0.5-1 μg/ml, BD Biosciences, San Jose, Calif.) at 37° C. for 72 hours and harvested as previously reported (Sumpter et al., 2008). This generalized protocol was used to measure T cell proliferation of CD4⁺ and CD8⁺ T cells following the various isolation methods and treatment conditions indicated. In MMP deficient parallel studies, MMP2−/−, MMP9−/−, MMP2/9−/− mice and littermate controls were cultured in the presence of anti-CD3 antibody for 72 h. In antigen-specific proliferation assays, OT-II transgenic and OT-I transgenic T cells were incubated with indicated concentrations of SB-3CT or vehicle control for 6 hours, washed three times in RPMI and cultured (1×10⁵/well) in the presence of OVA-pulsed (OTII: ova peptide and OT-I: SIINFEKL peptide) antigen presenting cells (APCs) for 72 hours. In the T cell suppressor assays, CD4⁺25− or CD4⁺25⁺ T cells isolated from C57BL/6 mice were incubated with the indicated concentrations of SB-3CT or vehicle control for 6 hours. The cells were washed three times in RPMI and added at varying ratios (treated: untreated) in co-culture with untreated CD4⁺25− T cells in the presence of γ-irradiated antigen presenting cells in 200 μl of cRPMI at 37° C. for 72 hours and harvested as previously reported (Sumpter et al., 2008).

Gelatin Zymography

Cell lysates and conditioned media supernatant were collected, concentrated to 4× and centrifuged to remove any cell debris, and stored at −80° C. prior to assay. Samples were then subjected to zymography as reported previously (Yoshida et al., 2007).

Cytokine Profiling by Quantitative RT PCR

Purified CD4⁺ T cells were incubated with the indicated concentrations of SB3-CT for 6 hours and then washed three times with RPMI 1640. Drug or vehicle-treated T cells were cultured (1×10⁶/ml) with anti-CD3 antibody (0.5 μg/ml) in cRPMI for 1-12 hours. Cells were collected and total RNA was isolated using an RNeasy RNA extraction kit (Qiagen, Inc., Valencia, Calif.) and mRNA expression levels were detected with PerfeCTa™ SYBR Green FastMix, Low ROX (Quanta Biosciences, Gaithersburg, Md.) on a Applied Biosystems 7500 according to the manufacturer's instructions. Each sample was normalized to murine β-actin. Primer sequences were designed and optimized using routine methodologies to specifically amplify each cytokine based on publicly available sequences.

Cytokine Profiling by Cytometric Bead Array (CBA)

Purified MMP9 deficient or SB-3CT-treated (10 μM) CD4⁺ T cells were incubated for 6 hours and then washed three times with RPMI 1640. MMP9 deficient or SB-3CT-treated T cells were cultured (1×10⁶/ml) with anti-CD3 antibody (0.5 μg/ml) in cRPMI for 1-12 hours. Supernatants were collected and cytokine protein levels were measured using the Mouse Inflammatory Cytokine Bead Array Kit (BD Biosciences, San Jose, Calif.) according to the manufacturer's instructions.

Intracellular Calcium Flux

Calcium flux was measured in CD4⁺ and CD8⁺ wild-type or MMP9 deficient or SB-3CT-treated (10 μM) T cells using the Fluo-4 NW Calcium Assay kit (Molecular Probes, Carlsbad, Calif.) in accord with the manufacturer's protocol. Cells were then stimulated with anti-CD3 antibody (10 μg/ml) and read in real time on a Molecular Devices FlexStation I (Sunnyvale, Calif.) for 300 seconds.

Cell Phenotyping of MMP9−/− T Cells

CD4⁺ and CD8⁺ T cells were isolated from wild-type and MMP9 deficient mice. Following the various treatment conditions, the cells were collected and washed in FACS buffer (10% BSA in PBS). Non-specific binding was blocked with FACS buffer supplemented with anti-CD16/anti-CD3 Ab (0.5 μg/well, eBioscience, San Diego, Calif.). Cells were then stained with anti-mouse CD4-FITC, CD8-PE, CD25-PE, CD40L-PE, CD44-PE, CD45RO-FITC, CD62L-APC, CD69-FITC, and CTLA-4-PE antibodies along with the corresponding isotype controls (all from eBioscience). After staining, cells were fixed in a 3% paraformaldehyde solution and read immediately on the flow cytometer. The data from 10,000 cells in the live gate were analyzed with a FACScan flow cytometer (Beckton Dickinson). FCS Express (DeNovo Software, Los Angeles, Calif.) was used for further analysis.

Activation of OT-I Thy1.1⁺ CD8⁺ T Cells and Adoptive Transfer Into CC10-OVA Mice

Lymph node and spleen were isolated from Thy1.1⁺ OT-I transgenic mice and splenic CD8⁺ T cells were isolated as stated above. OT-I Thy1.1⁺ CD8⁺ T cells were then treated with 10 μM of SB-3CT or the corresponding vehicle control (DMSO⁺ PEG) for 6 hours, followed by three washes in culture media. 5×10⁷ γ-irradiated wild-type splenocytes were cultured in 30 ml of 10% DMEM supplemented with 0.7 μg/ml of OVA peptide (SIINFEKL) for 5 min, followed by the addition of OT-1 Thy1.1⁺ CD8⁺ T cells (5×10⁶), anti-CD28 antibody (2 μg/ml), IL-2 (132.02 U/ml) and IL-12 (10 ng/ml). On day 3, the cells were split and supplemented with more IL-2 (25 U/ml) in a final volume of 30 ml. On day 5, cells were harvested and prepared for adoptive transfer into CC10-OVA mice. Cells were resuspended in PBS, and 7.5×10⁵ cells were intratracheally instilled into the lungs of CC10-OVA mice.

Identification of OT-I Thy1.1⁺ CD8⁺ T Cells in the Lung of CC10-OVA Mice Following Adoptive Transfer

The lungs of CC10-OVA mice were perfused and excised 10 days after adoptive transfer of SB-3CT- or vehicle treated OT-I Thy1.1⁺ CD8⁺ T cells. The lung was finely minced on ice, followed by a 60-90 minute digestion at 37° C. with collagenase/dispase (0.2 mg/ml of each) in RPMI medium with 5% fetal calf serum (FCS), in the presence of 25 μg/ml DNase. Cells were passed through a 70 μm cell strainer, washed, and lung lymphocytes were isolated by density centrifugation. Cells were resuspended in FACS buffer (10% BSA in PBS) and analyzed immediately on a FACScan flow cytometer (Beckton Dickinson). FCS Express (DeNovo Software, Los Angeles, Calif.) was used for further analysis.

Cell Subset Identification in BAL

BAL was collected from the lungs of wild-types and CC10 mice following adoptive transfer of vehicle and SB-3CT-treated OT-1 Tg T cells, by washing the mouse lung with 1.0 ml of sterile 1×PBS. Collected fluid was then centrifuged for 10 minutes at 2000 rpm. Cell pellets were resuspended in 200 μl of sterile 1×PBS. Cells were then stained with anti-GR1 antibody and analyzed immediately on a FACScan flow cytometer (Beckton Dickinson). FCS Express (DeNovo Software, Los Angeles, Calif.) was used for further analysis.

Histology

Lungs were perfused, inflated and fixed with neutral buffered formalin. The sections were then embedded in paraffin, sectioned, and stained with hematoxylin and eosin. Images were acquired at 20× using an Olympus microscope and DP12 digital camera (Olympus, Center Valley, Pa.).

Statistical Analysis

Data were analyzed by either 2-way Analysis of Variance (ANOVA) with paired t-test or nonparametric t-test using Prism 4 (GraphPad Software for Windows, San Diego, Calif.) or Microsoft Office Excel 2007 (Microsoft, Seattle, Wash.)

### REFERENCES

Baroja, M. L., L. Vijayakrishnan, E. Bettelli, P. J. Darlington, T. A. Chau, V. Ling, M. Collins, B. M. Carreno, J. Madrenas, and V. K. Kuchroo. 2002. Inhibition of CTLA-4 Function by the Regulatory Subunit of Serine/Threonine Phosphatase 2A. J Immunol 168:5070-5078. Belleguic, C., M. Corbel, N. Germain, H. Léna, E. Boichot, P. Delaval, and V. Lagente. 2002. Increased release of matrix metalloproteinase 2 and 9 in the plasma of acute severe asthmatic patients. Clinical & Experimental Allergy 32:217-223. Bourguignon, L. Y. W., H. Zhu, L. Shao, and Y.-W. Chen. 2001. CD44 Interaction with c-Src Kinase Promotes Cortactin-mediated Cytoskeleton Function and Hyaluronic Acid-dependent Ovarian Tumor Cell Migration. J. Biol. Chem. 276:7327-7336. Brown, S., M. M. Bernardo, Z. H. Li, L. P. Kotra, Y. Tanaka, R. Fridman, and S. Mobashery. 2000. Potent and selective mechanism-based inhibition of gelatinases. Journal of the American Chemical Society 122:6799-6800. Campbell, L. G., S. Ramachandran, W. Liu, J. M. Shipley, S. Itohara, J. G. Rogers, N. Moazami, R. M. Senior, and A. Jaramillo. 2005. Different Roles for Matrix Metalloproteinase-2 and Matrix Metalloproteinase-9 in the Pathogenesis of Cardiac Allograft Rejection. American Journal of Transplantation 5:517-528. Carbone, F., and M. Bevan. 1989. Induction of ovalbumin-specific cytotoxic T cells by in vivo peptide immunization. J. Exp. Med. 169:603-612. Chen, C., E. A. Rowell, R. M. Thomas, W. W. Hancock, and A. D. Wells. 2006. Transcriptional Regulation by Foxp3 Is Associated with Direct Promoter Occupancy and Modulation of Histone Acetylation. J. Biol. Chem. 281:36828-36834. Corry, D. B., A. Kiss, L.-Z. Song, L. Song, J. Xu, S.-H. Lee, Z. Werb, and F. Kheradmand. 2004. Overlapping and independent contributions of MMP2 and MMP9 to lung allergic inflammatory cell egression through decreased CC chemokines. FASEB J. 03-1412fje. Cosio, M. G., and A. Guerassimov. 1999. Chronic Obstructive Pulmonary Disease. Inflammation of Small Airways and Lung Parenchyma. Am. J. Respir. Crit. Care Med. 160:S21-25. Creemers, E. E. J. M., J. P. M. Cleutjens, J. F. M. Smits, and M. J. A. P. Daemen. 2001. Matrix Metalloproteinase Inhibition After Myocardial Infarction: A New Approach to Prevent Heart Failure? Circ Res 89:201-210. Crouch, E. 1990. Pathobiology of pulmonary fibrosis. Am J Physiol Lung Cell Mol Physiol 259:L159-184. Dell'Agli, M., G. V. Galli, E. Bosisio, and M. D'Ambrosio. 2009. Inhibition of NF-kB and metalloproteinase-9 expression and secretion by parthenolide derivatives. Bioorganic & Medicinal Chemistry Letters 19:1858-1860. Feng, C., K. J. Woodside, B. A. Vance, D. El-Khoury, M. Canelles, J. Lee, R. Gress, B. J. Fowlkes, E. W. Shores, and P. E. Love. 2002. A potential role for CD69 in thymocyte emigration. Int. Immunol. 14:535-544. Fernandez, F. G., L. G. Campbell, W. Liu, J. M. Shipley, S. Itohara, G. A. Patterson, R. M. Senior, T. Mohanakumar, and A. Jaramillo. 2005. Inhibition of obliterative airway disease development in murine tracheal allografts by matrix metalloproteinase-9 deficiency. American Journal of Transplantation 5:671-683. Forbes, C., Q. Shi, J. F. Fisher, M. Lee, D. Hesek, L. I. Llarrull, M. Toth, M. Gossing, R. Fridman, and S. Mobashery. 2009. Active site ring-opening of a thiirane moiety and picomolar inhibition of gelatinases. Chem Biol Drug Des 74:527-534. Goetzl, E., M. Banda, and D. Leppert. 1996. Commentary: Matrix metalloproteinases in immunity. J Immunol 156:1-4. Graesser, D., S. Mahooti, and J. A. Madri. 2000. Distinct roles for matrix metalloproteinase-2 and [alpha]-4 integrin in autoimmune T cell extravasation and residency in brain parenchyma during experimental autoimmune encephalomyelitis. Journal of Neuroimmunology 109:121-131. Greenlee, K. J., D. B. Corry, D. A. Engler, R. K. Matsunami, P. Tessier, R. G. Cook, Z. Werb, and F. Kheradmand. 2006. Proteomic Identification of In vivo Substrates for Matrix Metalloproteinases 2 and 9 Reveals a Mechanism for Resolution of Inflammation. J Immunol 177:7312-7321. Hall, S. R., and J. Rhodes. 2001. Schiff base-mediated co-stimulation primes the T-cell-receptor-dependent calcium signalling pathway in CD4 T cells. Immunology 104:50-57. Hubner, R. H., S. Meffert, U. Mundt, H. Bottcher, S. Freitag, N. E. El Mokhtari, T. Pufe, S. Hirt, U. R. Folsch, and B. Bewig. 2005 Matrix metalloproteinase-9 in bronchiolitis obliterans syndrome after lung transplantation. European Respiratory Journal 25:494-501. Me-Sasaki, J., T. Sasaki, and J. M. Penninger. 2003. CD45 Regulated Signaling Pathways. Current Topics in Medicinal Chemistry 3:783-796. Ivetic, A., and A. J. Ridley. 2004. The telling tail of L-selectin. Biochem. Soc. Trans. 32:1118-1121. Kelly, K. E., M. I. Hertz, and D. L. Mueller. 1998. T-Cell and Major Histocompatibility Complex Requirements for Obliterative Airway Disease in Heterotopically Transplanted Murine Tracheas1. Transplantation 66:764-771. Kwan, J. A., C. J. Schulze, W. Wang, H. Leon, M. Sariahmetoglu, M. Sung, J. Sawicka, D. E. Sims, G. Sawicki, and R. Schulz. 2004. Matrix metalloproteinase-2 (MMP-2) is present in the nucleus of cardiac myocytes and is capable of cleaving poly (ADP-ribose) polymerase PARP in vitro. FASEB J. 02-1202fje. Lee, S.-M., B. Gao, and D. Fang. 2008. FoxP3 maintains Treg unresponsiveness by selectively inhibiting the promoter DNA-binding activity of AP-1. Blood 111:3599-3606. Leo, E., K. Welsh, S.-i. Matsuzawa, J. M. Zapata, S. Kitada, R. S. Mitchell, K. R. Ely, and J. C. Reed. 1999. Differential Requirements for Tumor Necrosis Factor Receptor-associated Factor Family Proteins in CD40-mediated Induction of NF-kappa B and Jun N-terminal Kinase Activation. J. Biol. Chem. 274:22414-22422. Marson, A., K. Kretschmer, G. M. Frampton, E. S. Jacobsen, J. K. Polansky, K. D. MacIsaac, S. S. Levine, E. Fraenkel, H. von Boehmer, and R. A. Young. 2007. Foxp3 occupancy and regulation of key target genes during T-cell stimulation. Nature 445:931-935. Medoff, B. D., E. Seung, J. C. Wain, T. K. Means, G. S. V. Campanella, S. A. Islam, S. Y. Thomas, L. C. Ginns, N. Grabie, A. H. Lichtman, A. M. Tager, and A. D. Luster. 2005. BLT1-mediated T cell trafficking is critical for rejection and obliterative bronchiolitis after lung transplantation. J. Exp. Med. 202:97-110. Nagase, H., and J. F. J. Woessner. 1999. Matrix Metalloproteinases. J. Biol. Chem. 274:21491-21494. Neuringer, I. P., R. B. Mannon, T. M. Coffman, M. Parsons, K. Burns, J. R. Yankaskas, and R. M. Aris. 1998. Immune Cells in a Mouse Airway Model of Obliterative Bronchiolitis. Am. J. Respir. Cell Mol. Biol. 19:379-386. Neuringer, I. P., S. P. Walsh, R. B. Mannon, S. Gabriel, and R. M. Aris. 2000. Enhanced T cell cytokine gene expression in mouse airway obliterative bronchiolitis. Transplantation 69:399-405. Opdenakker, G., P. Van-den-Steen, and J. Van-Damme. 2001a. Gelatinase B: a tuner and amplifier of immune functions. Trends in Immunology 22:571-579. Opdenakker, G., P. E. Van den Steen, B. Dubois, I. Nelissen, E. Van Coillie, S. Masure, P. Proost, and J. Van Damme. 2001b. Gelatinase B functions as regulator and effector in leukocyte biology. J Leukoc Biol 69:851-859. Oviedo-Orta, E., A. Bermudez-Fajardo, S. Karanam, U. Benbow, and A. G. Newby. 2008. Comparison of MMP-2 and MMP-9 secretion from T helper 0, 1 and 2 lymphocytes alone and in coculture with macrophages. Immunology 124:42-50. Pan, F., H. Yu, E. V. Dang, J. Barbi, X. Pan, J. F. Grosso, D. Jinasena, S. M. Sharma, E. M. McCadden, D. Getnet, C. G. Drake, J. O. Liu, M. G. Ostrowski, and D. M. Pardoll. 2009. Eos Mediates Foxp3-Dependent Gene Silencing in CD4⁺Regulatory T Cells. Science 325:1142-1146. Rosenblum, G., S. Meroueh, M. Toth, J. F. Fisher, R. Fridman, S. Mobashery, and I. Sagi. 2007. Molecular Structures and Dynamics of the Stepwise Activation Mechanism of a Matrix Metalloproteinase Zymogen: Challenging the Cysteine Switch Dogma. Journal of the American Chemical Society 129:13566-13574. Sandler, C., E. Ekokoski, K. A. Lindstedt, P. J. Vainio, M. Finel, T. Sorsa, P. T. Kovanen, L. M. Golub, and K. K. Eklund. 2005. Chemically modified tetracycline (CMT)-3 inhibits histamine release and cytokine production in mast cells: possible involvement of protein kinase C. Inflammation Research 54:304-312. Shapiro, S. D., and R. M. Senior. 1999. Matrix Metalloproteinases. Matrix Degradation and More. Am. J. Respir. Cell Mol. Biol. 20:1100-1102. Shilling, R. A., B. S. Clay, A. G. Tesciuba, E. L. Berry, T. Lu, T. V. Moore, H. S. Bandukwala, J. Tong, J. V. Weinstock, R. A. Flavell, T. Horan, S. K. Yoshinaga, A. A. Welcher, J. L. Cannon, and A. I. Sperling. CD28 and ICOS play complementary non-overlapping roles in the development of Th2 immunity in vivo. Cellular Immunology In Press, Corrected Proof: Si-Tayeb, K., A. Monvoisin, C. Mazzocco, S. Lepreux, M. Decossas, G. Cubel, D. Taras, J.-F. Blanc, D. R. Robinson, and J. Rosenbaum. 2006. Matrix Metalloproteinase 3 Is Present in the Cell Nucleus and Is Involved in Apoptosis. Am J Pathol 169:1390-1401. Stauber, D. J., E. W. Debler, P. A. Horton, K. A. Smith, and I. A. Wilson. 2006. Crystal structure of the IL-2 signaling complex: Paradigm for a heterotrimeric cytokine receptor. Proceedings of the National Academy of Sciences of the United States of America 103:2788-2793. Sternlicht, M. D., and Z. Werb. 2001. How matrix metalloproteinases regulate cell behavior. Annual Review of Cell and Developmental Biology 17:463-516. Stripp, B., P. Sawaya, D. Luse, K. Wikenheiser, S. Wert, J. Huffman, D. Lattier, G. Singh, S. Katyal, and J. Whitsett. 1992. cis-acting elements that confer lung epithelial cell expression of the CC10 gene. J. Biol. Chem. 267:14703-14712. Sumpter, T. L., K. K. Payne, and D. S. Wilkes. 2008. Regulation of the NFAT pathway discriminates CD4⁺ CD25⁺ regulatory T cells from CD4⁺ CD25− helper T cells. J Leukoc Biol 83:708-717. Thornton, A. M., and E. M. Shevach. 1998. CD4⁺ CD25⁺ Immunoregulatory T Cells Suppress Polyclonal T Cell Activation In vitro by Inhibiting Interleukin 2 Production. J. Exp. Med. 188:287-296. Toth, M., M. M. Bernardo, D. C. Gervasi, P. D. Soloway, Z. Wang, H. F. Bigg, C. M. Overall, Y. A. DeClerck, H. Tschesche, M. L. Chem., S. Brown, S. Mobashery, and R. Fridman. 2000. Tissue inhibitor of metalloproteinase (TIMP)-2 acts synergistically with synthetic matrix metalloproteinase (MMP) inhibitors but not with TIMP-4 to enhance the (Membrane type 1)-MMP-dependent activation of pro-MMP-2. J Biol Chem 275:41415-41423. Wu, Y., M. Borde, V. Heissmeyer, M. Feuerer, A. D. Lapan, J. C. Stroud, D. L. Bates, L. Guo, A. Han, S. F. Ziegler, D. Mathis, C. Benoist, L. Chen, and A. Rao. 2006. FOXP3 Controls Regulatory T Cell Function through Cooperation with NFAT. Cell 126:375-387. Yoshida, H., H. Nishina, H. Takimoto, L. E. M. Marengere, A. G. Wakeham, D. Bouchard, Y.-Y. Kong, T. Ohteki, A. Shahinian, M. Bachmann, P. S. Ohashi, J. M. Penninger, G. R. Crabtree, and T. W. Mak. 1998. The Transcription Factor NF-ATc1 Regulates Lymphocyte Proliferation and Th2 Cytokine Production. Immunity 8:115-124. Yoshida, S., T. Iwata, M. Chiyo, G. N. Smith, B. H. Foresman, E. A. Mickler, K. M. Heidler, O. W. Cummings, T. Fujisawa, D. D. Brand, A. Baker, and D. S. Wilkes. 2007. Metalloproteinase Inhibition Has Differential Effects on Alloimmunity, Autoimmunity, and Histopathology in the Transplanted Lung. Transplantation 83:799-808. Zitt, C., B. Strauss, E. G. Schwarz, N. Spaeth, G. Rast, A. Hatzelmann, and M. Hoth. 2004. Potent Inhibition of Ca2⁺ Release-activated Ca2⁺ Channels and T-lymphocyte Activation by the Pyrazole Derivative BTP2. J. Biol. Chem. 279:12427-12437.

All of the above U.S. patents, U.S. patent application publications, U.S. patent applications, foreign patents, foreign patent applications and non-patent publications referred to in this specification and/or listed in the Application Data Sheet, including but not limited to U.S. Provisional Application No. 61/152,512, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-8. (canceled)

9. A method for inhibiting an immune response against a collagen in a patient in need thereof comprising, administering to the patient a therapeutically effective amount of a compound of Formula I: 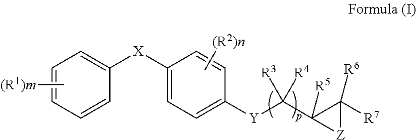
wherein:
m is 0, 1, 2, 3, 4 or 5;
n is 0, 1, 2, 3, 4 or 5;
p is 1, 2 or 3;
X is —O—, —S—, —CH₂— or a direct bond;
Y is —C(O)— or —S(O)₂—,
Z is —O— or —S—;
R¹ at each occurrence is the same or different and independently alkyl, alkenyl, aralkyl, haloalkyl, halogen, —OR⁸ or —NR⁹R¹⁰;
R² at each occurrence is the same or different and independently alkyl, alkenyl, aralkyl, haloalkyl, halogen, —OR⁸ or —NR⁹R¹⁰;
R³ and R⁴ are each the same or different and independently hydrogen or alkyl;
R⁵, R⁶ and R⁷ are each the same or different and independently hydrogen or alkyl;
R⁸ is hydrogen, alkyl, alkenyl, or aryl; and
R⁹ and R¹⁰ are each the same or different and independently hydrogen or alkyl;
or a pharmaceutically acceptable salt thereof.

10. The method of claim 9 wherein the compound of formula (I) is a compound of formula (Ia):

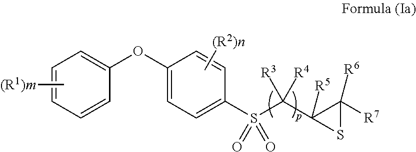

11. The method of claim 9 wherein the compound is SB-3CT

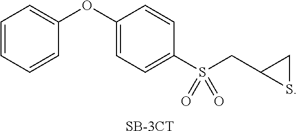

12. The method of claim 9 wherein the compound of formula (I) is a compound of formula (Ib):

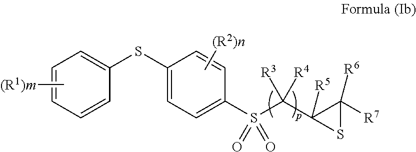

13. The method of claim 9 wherein the compound of formula (I) is a compound of formula (Ic):

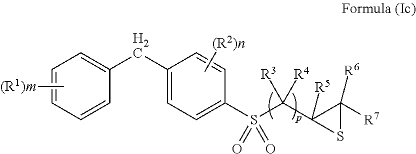

14. The method of claim 9 wherein the transplant patient is a lung transplant patient.

15.-21. (canceled)

22. A method for inhibiting an immune response in a patient in need thereof comprising, administering to the patient a therapeutically effective amount of a compound of Formula I: 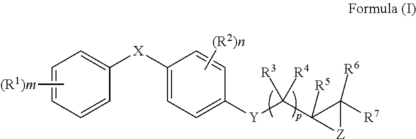
wherein:
m is 0, 1, 2, 3, 4 or 5;
n is 0, 1, 2, 3, 4 or 5;
p is 1, 2 or 3;
X is —O—, —S—, —CH₂— or a direct bond;
Y is —C(O)— or —S(O)₂—,
Z is —O— or —S—;
R¹ at each occurrence is the same or different and independently alkyl, alkenyl, aralkyl, haloalkyl, halogen, —OR⁸ or —NR⁹R¹⁰;
R² at each occurrence is the same or different and independently alkyl, alkenyl, aralkyl, haloalkyl, halogen, —OR⁸ or —NR⁹R¹⁰;
R³ and R⁴ are each the same or different and independently hydrogen or alkyl;
R⁵, R⁶ and R⁷ are each the same or different and independently hydrogen or alkyl;
R⁸ is hydrogen, alkyl, alkenyl, or aryl; and
R⁹ and R¹⁰ are each the same or different and independently hydrogen or alkyl;
or a pharmaceutically acceptable salt thereof.

23. The method of claim 22 wherein the patient in need thereof has an autoimmune disease selected from the group consisting of alloimmune-induced autoimmunity post organ transplant, collagen vascular diseases and multiple sclerosis.

24. The method of claim 22 wherein the patient in need thereof has asthma.

25. The method of claim 22 wherein the patient in need thereof has a T cell mediated pulmonary disease.

26. The method of claim 22 wherein the immune response comprises a CD8+ T cell response.

27. The method of claim 26 wherein the CD8+ T cell response is an antigen-specific response.

28. The method of claim 22 wherein the immune response comprises a CD4+ T cell response.

29. The method of claim 28 wherein the CD4+ T cell response is an antigen-specific response.

30. The method of claim 22 wherein regulatory T cells are not inhibited by the compound of Formula I.

31. The method of claim 22 wherein the patient is a solid organ transplant patient.

32.-34. (canceled)

35. A composition comprising an effective amount of a compound of Formula I in combination with an immunosuppressant wherein the effective dosage of the immunosuppressant is reduced as compared to the effective dosage normally used in the absence of the compound of Formula I.

36.-39. (canceled)

40. The method of claim 39 wherein the patient is a patient in need of a lung transplant or a lung transplant patient.

41. The method of claim 39 wherein the collagen is Type V collagen.

42. The method of claim 41, comprising administering to the patient in need thereof an effective amount of a compound of Formula I in combination with an effective amount of Type V collagen, or a tolerizing fragment thereof.

43. The method of claim 42, wherein the Type V collagen, or a tolerizing fragment thereof, is administered orally or intravenously.
